(19) United States
(12) Patent Application Publication
(10) Pub. No.: US 2009/0247545 A1
(43) Pub. Date: Oct. 1, 2009

(54) SUBSTITUTED OXAZOLIDINONE DERIVATIVES

(76) Inventors: Mukund Keshao Gurjar, Pune (IN); Madhusudan Nagorao Deshmukh, Pune (IN); Anita Mehta, Plainfield, IL (US); Sudershan Kumar Arora, Pune (IN); Ashok Rattan, Haryana (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 11/577,083

(22) PCT Filed: Dec. 28, 2004
(86) PCT No.: PCT/IB04/04278

§ 371 Date: Jun. 8, 2009

(30) Foreign Application Priority Data
Oct. 11, 2004 (IN) 1962/DEL/2004

(51) Int. Cl. A61K 31/496; C07D 413/10; A61P 31/04

(52) U.S. Cl. 514/254.02; 544/369

(57) ABSTRACT

The present invention provides substituted oxazolidinone derivatives, which can be used as antimicrobial agents. Compounds disclosed can be used for the treatment or prevention of a condition caused by or contributed to by bacteria, such as, inter alia, multiply-resistant Staphylococci, Streptococci, Enterococci, Bacterioides spp., Clostridium spp., Mycobacterium spp. Bacillus spp., Corynebacterium spp. Heptoslreptacoccus spp. Listeria spp., Legionella spp., Haemophilus influenza, Moraxella, Eschericia faecalis, and Eschericia coli. Processes for the preparation of disclosed compounds, pharmaceutical compositions thereof, and method of treating microbial infection are provided.

### FIELD OF INVENTION

The present invention provides substituted oxazolidinone derivatives, which can be used as antimicrobial agents. Compounds disclosed can be used for the treatment or prevention of a condition caused by or contributed to by bacteria Processes for the preparation of disclosed compounds, pharmaceutical compositions thereof, and methods of treating microbial infection are provided.

### BACKGROUND OF THE INVENTION

The international microbiological community continues to express serious concern that the evolution of antibiotic resistance could result in strains against which currently available antibacterial agents will be ineffective. Gram-positive pathogens, for example, Staphylococci, Enterococci and Streptococci, are particularly important because of the development of resistant strains, which are both difficult to treat and eradicate from the hospital environment once established. Examples of such strains include methecin-resistant Staphylococcus aureus (MRSA), methecin-resistant Staphylococcus epidermidis (MRSE), methecin-resistant coagulase negative Staphylococcus (MRCNS), glycopeptide intermediate-resistant Staphylococcus aureus (GISA), vancomycin-resistant Enterococci (VRE), penicillin-resistant Streptococcus pneumoniae and multiply-resistant Enterococcus faecium. Increasing resistance also is appearing towards agents, such as β-Lactams, quinolones and macrolides, used for the treatment of upper respiratory tract infection, also caused by certain gram-negative strains that include H. Influenza and M. Catarrhalis.

Oxazolidinones are a new class of synthetic antimicrobial agents, which have been disclosed as being effective agents against gram-positive pathogens by inhibiting a very early stage protein synthesis. It has been reported that oxazolidinones inhibit the formation of ribosomal initiation complex involving 30S and 50S ribosomes leading to prevention of initiation complex formation. These compounds have a mechanism of action that allow them to be active against pathogens resistant to other clinically useful antibiotics. Examples of oxazolidinones include linezolid and eperezolid.

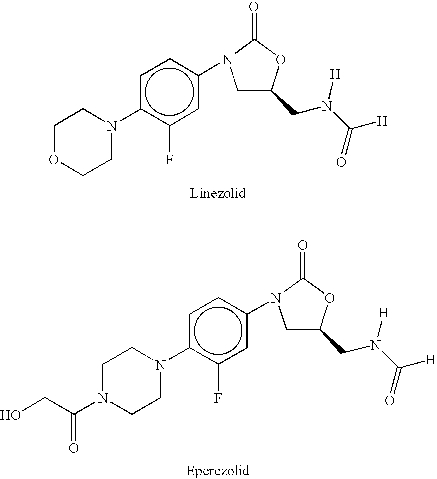

Substituted phenyl oxazolidinones have been disclosed as being useful antimicrobial agents, effective against a number of human and veterinary pathogens including gram-positive bacteria, which includes multiply-resistant Staphylococci, Streptococci and Enterococci, as well as anaerobic organisms such as Bacterioides spp., Clostridium spp., and acid-fast organisms, which include Mycobacterium tuberculosis, Mycobacterium avium and other Mycobacterium spp.

Oxazolidinone derivatives also have been disclosed as being useful antimicrobials agents, effective against a number of human and veterinary pathogens, including gram-positive strains, such as the resistant strains of Staphylococci and Enterococci.

Although antimicrobial agents are available, there remains a need for novel antimicrobial agents that are effective against pathogens having antibiotic resistance.

### SUMMARY OF THE INVENTION

The present invention results from the recognition that particular substituted oxazolidinone derivatives can be useful antimicrobial agents.

It is also an aspect of the invention to provide for processes for the synthesis of such substituted oxazolidinone derivatives.

It is a further aspect of the invention to provide pharmaceutical compositions containing such substituted oxazolidinone derivatives and which also may contain pharmaceutically acceptable carriers, excipients or diluents.

It is a still further aspect of the invention to provide methods of the treatment or prevention of a condition caused by or contributed to by microbial infection.

The present invention encompasses pharmaceutically acceptable salts, pharmaceutically acceptable solvates, enantiomers, diastereomers, N-oxides or polymorphs of these compounds having same type of activity.

In one embodiment, there are provided compounds having the structure of Formula I:

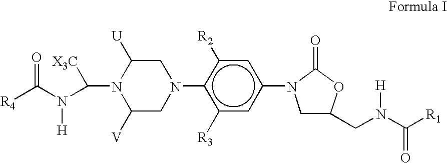

and its pharmaceutically acceptable salts, pharmaceutically acceptable solvates, enantiomers, diasteromers, N-oxides or polymorphs.
R₁ and R₄ can be hydrogen; optionally substituted alkyl, alkenyl, alkynyl, or cycloalkyl (each of which may be substituted with one or more of halogen, hydroxyl or alkoxy); or optionally substituted aryl or heterocycle (each of which may be substituted with one or more of halogen, hydroxy, mercapto, alkoxy, alkyl, acyl, acyloxy, haloalkyl, amino, cyano, nitro, thio, or thioalkyl).
R₂ and R₃ can be hydrogen, halogen, alkyl, or haloalkyl.
U and V can be hydrogen, alkyl, or haloalkyl.
X can be halogen.

Particular compounds of Formula I may have R₁ as [alkyl optionally substituted with halogen] in particular methyl or trifluoromethyl). Other particular compounds of Formula I may have R₃ as halogen in particular fluorine. In other particular compounds of Formula I, U may be alkyl preferably methyl. In yet other particular compounds, X can be halogen particularly, fluorine, chlorine or bromine. Other particular compounds of Formula I may have R₄ as alkyl or aryl optionally trichloromethyl substituted with halogen for example, methyl, trifluoromethyl, phenyl or 2,4-dichloro-5-fluorophenyl).

Other particular compounds of Formula I include:
N-((3-(4-(3-Fluoro-4(1′-formamido-2′,2′,2′-trichloroethyl)-1-piperazinyl)phenyl)-2-oxo-5-oxazolidinyl)methyl)-acetamide (Compound No. 1),
N-((3-(4-(3-Fluoro-4(1′-acetamido-2′,2′,2′-trichloroethyl)-1-piperazinyl)phenyl)-2-oxo-5-oxazolidinyl)methyl)-acetamide (Compound No. 2),
N-((3-(4-(3-Fluoro-4(1′-trichloroacetamido-2′,2′,2′-trichloroethyl)-1-piperazinyl)phenyl)-2-oxo-5-oxazolidinyl)methyl)-acetamide (Compound No. 3),
N-((3-(4-(3-Fluoro-4(1′-trifluoroacetamido-2′,2′,2-trichloroethyl)-1-piperazinyl)phenyl)-2-oxo-5-oxazolidinyl)methyl)-acetamide (Compound No. 4),
N-((3-(4-(3-Fluoro-4(1′-formamido-2′,2′,2′-tribromoethyl)-1-piperazinyl)phenyl)-2-oxo-5-oxazolidinyl)methyl)-acetamide (Compound No. 5),
N-((3-(4-(3-Fluoro-4(1′-acetamido-2′,2′,2′-tribromoethyl)-1-piperazinyl)phenyl)-2-oxo-5-oxazolidinyl)methyl)-acetamide (Compound No. 6),
N-((3-(4-(3-Fluoro-4(1′-trifluoroacetamido-2′,2′,2′-tribromoethyl)-1-piperazinyl)phenyl)-2-oxo-5-oxazolidinyl)methyl)-acetamide (Compound No. 7),
N-((3-(4-(3-Fluoro-4(1′-benzamido-2′,2′,2′-trichloroethyl)-1-piperazinyl)phenyl)-2-oxo-5-oxazolidinyl)methyl)-acetamide (Compound No. 8),
N-((3-(4-(3-Fluoro-4(1′-dichloro-5-fluorobenzamido-2′,2′,2′-trichloroethyl)-1-piperazinyl)phenyl)-2-oxo-5-oxazolidinyl)methyl)-acetamide (Compound No. 9),
N-((3-(4-(3-Fluoro-4(1′-benzamido-2′,2′,2′-tribromoethyl)-1-piperazinyl)phenyl)-2-oxo-5-oxazolidinyl)methyl)-acetamide (Compound No. 10),
N-((3-(4-(3-Fluoro-4(1′-2,4-dichloro-5-fluorobenzamido-2′,2′,2′-tribromoethyl)-1-piperazinyl)phenyl)-2-oxo-5-oxazolidinyl)methyl)-acetamide (Compound No. 11),
N-((3-(4-(3-Fluoro-4(1′-formamido-2′,2′,2′-trifluoroethyl)-1-piperazinyl)phenyl)-2-oxo-5-oxazolidinyl)methyl)-acetamide (Compound No. 12),
(S)—N-((3-(4-(3-Fluoro-4(1′-formamido-2′,2′,2′-trichloroethyl)-1-piperazinyl)phenyl)-2-oxo-5-oxazolidinyl)methyl)-acetamide (Compound No. 13),
(S)—N-((3-(4-(3-Fluoro-4(1′-acetamido-2′,2′,2′-trichloroethyl)-1-piperazinyl)phenyl)-2-oxo-5-oxazolidinyl)methyl)-acetamide (Compound No. 14),
(S)—N-((3-(4-(3-Fluoro-4(1′-trichloroacetamido-2′,2′,2′-trichloroethyl)-1-piperazinyl)phenyl)-2-oxo-5-oxazolidinyl)methyl)-acetamide (Compound No. 15),
(S)—N-((3-(4-(3-Fluoro-4(1′-trifluoroacetamido-2′,2′,2′-trichloroethyl)-1-piperazinyl)phenyl)-2-oxo-5-oxazolidinyl)methyl)-acetamide (Compound No. 16),
(S)—N-((3-(4-(3-Fluoro-4(1′-formamido-2′,2′,2′-tribromoethyl)-1-piperazinyl)phenyl)-2-oxo-5-oxazolidinyl)methyl)-acetamide (Compound No. 17),
(S)—N-((3-(4-(3-Fluoro-4(1′-acetamido-2′,2′,2′-tribromoethyl)-1-piperazinyl)phenyl)-2-oxo-5-oxazolidinyl)methyl)-acetamide (Compound No. 18),
(S)—N-((3-(4-(3-Fluoro-4(1′-trichloroacetamido-2′,2′,2′-tribromoethyl)-1-piperazinyl)phenyl)-2-oxo-5-oxazolidinyl)methyl)-acetamide (Compound No. 19),
(S)—N-((3-(4-(3-Fluoro-4(1′-trifluoroacetamido-2′,2′,2′-tribromoethyl)-1-piperazinyl)phenyl)-2-oxo-5-oxazolidinyl)methyl)-acetamide (Compound No. 20),
(S)—N-((3-(4-(3-Fluoro-4(1′-formamido-2′,2′,2′-trifluoroethyl)-1-piperazinyl)phenyl)-2-oxo-5-oxazolidinyl)methyl)-acetamide (Compound No. 21),
(S)—N-((3-(4-(3-Fluoro-4(1′-acetamido-2′,2′,2′-trifluoroethyl)-1-piperazinyl)phenyl)-2-oxo-5-oxazolidinyl)methyl)-acetamide (Compound No. 22),
(S)—N-((3-(4-(3-Fluoro-4(1′-formamido-2′,2′,2′-trichloroethyl)-1-piperazinyl)phenyl)-2-oxo-5-oxazolidinyl)methyl)-trifluoroacetamide (Compound No. 23),
(S)—N-((3-(4-(3-Fluoro-4(1′-acetamido-2′,2′,2′-trichloroethyl)-1-piperazinyl)phenyl)-2-oxo-5-oxazolidinyl)methyl)-trifluoroacetamide (Compound No. 24),
(S)—N-((3-(4-(3-Fluoro-4(1′-trichloroacetamido-2′,2′,2′-trichloroethyl)-1-piperazinyl)phenyl)-2-oxo-5-oxazolidinyl)methyl)-trifluoroacetamide (Compound No. 25),
(S)—N-((3-(4-(3-Fluoro-4(1′-trifluoroacetamido-2′,2′,2′-trichloroethyl)-1-piperazinyl)phenyl)-2-oxo-5-oxazolidinyl)methyl)-trifluoroacetamide (Compound No. 26),
(S)—N-((3-(4-(3-Fluoro-4(1′-formamido-2′,2′,2′-tribromoethyl)-1-piperazinyl)phenyl)-2-oxo-5-oxazolidinyl)methyl)-trifluoroacetamide (Compound No. 27),
(S)—N-((3-(4-(3-Fluoro-4(1′-acetamido-2′,2′,2′-tribromoethyl)-1-piperazinyl)phenyl)-2-oxo-5-oxazolidinyl)methyl)-trifluoroacetamide (Compound No. 28),
(S)—N-((3-(4-(3-Fluoro-4(1′-trichloroacetamido-2′,2′,2′-tribromoethyl)-1-piperazinyl)phenyl)-2-oxo-5-oxazolidinyl)methyl)-trifluoroacetamide (Compound No. 29),
(S)—N-((3-(4-(3-Fluoro-4(1′-trifluoroacetamido-2′,2′,2′-tribromoethyl)-1-piperazinyl)phenyl)-2-oxo-5-oxazolidinyl)methyl)-trifluoroacetamide (Compound No. 30),
(S)—N-((3-(4-(3-Fluoro-4(1′-formamido-2′,2′,2′-trifluoroethyl)-1-piperazinyl)phenyl)-2-oxo-5-oxazolidinyl)methyl)-trifluoroacetamide (Compound No. 31),
(S)—N-((3-(4-(3-Fluoro-4(1′-acetamido-2′,2′,2′-trifluoroethyl)-1-piperazinyl)phenyl)-2-oxo-5-oxazolidinyl)methyl)-trifluoroacetamide (Compound No. 32),
(S)—N-((3-(4-(3-Fluoro-4(1′-trichloroacetamido-2′,2′,2′-trifluoroethyl)-1-piperazinyl)phenyl)-2-oxo-5-oxazolidinyl)methyl)-trifluoroacetamide (Compound No. 33),
(S)—N-((3-(4-(3-Fluoro-4-(1′-formamido-2′,2′,2′-trichloroethyl)-3-methyl-1-piperazinyl)phenyl)-2-oxo-5-oxazolidinyl)methyl)-acetamide (Compound No. 34),
(S)—N-((3-(4-(3-Fluoro-4-(1′-acetamido-2′,2′,2′-trichloroethyl)-3-methyl-1-piperazinyl)phenyl)-2-oxo-5-oxazolidinyl)methyl)-acetamide (Compound No. 35),
(S)—N-((3-(4-(3-Fluoro-4-(1′-trichloroacetamido-2′,2′,2′-trichloroethyl)-3-methyl-1-piperazinyl)phenyl)-2-oxo-5-oxazolidinyl)methyl)-acetamide (Compound No. 36),
(S)—N-([3-(4-(3-Fluoro-4-(1′-trifluoroacetamido-2′,2′,2′-trichloroethyl)-3-methyl-1-piperazinyl)phenyl)-2-oxo-5-oxazolidinyl)methyl)-acetamide (Compound No. 37),
(S)—N-((3-(4-(3-Fluoro-4-(1′-formamido-2′,2′,2′-tribromoethyl)-3-methyl-1-piperazinyl)phenyl)-2-oxo-5-oxazolidinyl)methyl)-acetamide (Compound No. 38),
(S)—N-((3-(4-(3-Fluoro-4-(1′-acetamido-2′,2′,2′-tribromoethyl)-3-methyl-1-piperazinyl)phenyl)-2-oxo-5-oxazolidinyl)methyl)-acetamide (Compound No. 39),
(S)—N-((3-(4-(3-Fluoro-4-(1′-trichloroacetamido-2′,2′,2′-tribromoethyl)-3-methyl-1-piperazinyl)phenyl)-2-oxo-5-oxazolidinyl)methyl)-acetamide (Compound No. 40),
(S)—N-((3-(4-(3-Fluoro-4-(1′-trifluoroacetamido-2′,2′,2′-tribromoethyl)-3-methyl-1-piperazinyl)phenyl)-2-oxo-5-oxazolidinyl)methyl)-acetamide (Compound No. 41),
(S)—N-((3-(4-(3-Fluoro-4-(1′-formamido-2′,2′,2′-trifluoroethyl)-3-methyl-1-piperazinyl)phenyl)-2-oxo-5-oxazolidinyl)methyl)-acetamide (Compound No. 42),
(S)—N-((3-(4-(3-Fluoro-4-(1′-acetamido-2′,2′,2′-trifluorethyl)-methyl-1-piperazinyl)phenyl)-2-oxo-5-oxazolidinyl)methyl)-acetamide (Compound No. 43),
(S)—N-((3-(4-(3-Fluoro-4-(1′-trichloroacetamido-2′,2′,2′-trifluoroethyl)-3-methyl-1-piperazinyl)phenyl)-2-oxo-5-oxazolidinyl)methyl)-acetamide (Compound No. 44),
(S)—N-((3-(4-(3-Fluoro-4-(1′-formamaido-2′,2′,2′-tricholroethyl)-3-methyl-1-piperazinyl)phenyl)-2-oxo-5-oxazolidinyl)methyl)-trifluoroacetamide (Compound No. 45),
(S)—N-((3-(4-(3-Fluoro-4-(1′-acetamido-2′,2′,2′-trichloroethyl)-3-methyl-1-piperazinyl)phenyl)-2-oxo-5-oxazolidinyl)methyl)-trifluoroacetamide (Compound No. 46),
(S)—N-((3-(4-(3-Fluoro-4-(1′-trichloroacetamido-2′,2′,2′-trichloroethyl)-3-methyl-1-piperazinyl)phenyl)-2-oxo-5-oxazolidinyl)methyl)-trifluoroacetamide (Compound No. 47),
(S)—N-((3-(4-(3-Fluoro-4-(1′-trifluoroacetamido-2′,2′,2′-trichloroethyl)-3-methyl-1-piperazinyl)phenyl)-2-oxo-5-oxazolidinyl)methyl)-trifluoroacetamide (Compound No. 48),
(S)—N-((3-(4-(3-Fluoro-4-(1′-formamido-2′,2′,2′-tribromoethyl)-methyl-1-piperazinyl)phenyl)-2-oxo-5-oxazolidinyl)methyl)-trifluoroacetamide (Compound No. 49),
(S)—N-((3-(4-(3-Fluoro-4-(1′-acetamido-2′,2′,2′-tribromoethyl)-3-methyl-1-piperazinyl)phenyl)-2-oxo-5-oxazolidinyl)methyl)-trifluoroacetamide (Compound No. 50),
(S)—N-((3-(4-(3-Fluoro-4-(1′-trichloroacetamido-2′,2′,2′-tribromoethyl)-3-methyl-1-piperazinyl)phenyl)-2-oxo-5-oxazolidinyl)methyl)-trifluoroacetamide (Compound No. 51),
(S)—N-((3-(4-(3-Fluoro-4-(1′-trifluoroacetamido-2′,2′,2′-tribromoethyl)-3-methyl-1-piperazinyl)phenyl)-2-oxo-5-oxazolidinyl)methyl)-trifluoroacetamide (Compound No. 52),
(S)—N-((3-(4-(3-Fluoro-4-(1′-formamido-2′,2′,2′-trifluoroethyl)-3-methyl-1-piperazinyl)phenyl)-2-oxo-5-oxazolidinyl)methyl)-trifluoroacetamide (Compound No. 53) or
(S)—N-((3-(4-(3-Fluoro-4-(1′-acetamido-2′,2′,2′-trifluoroethyl)-3-methyl-1-piperazinyl)phenyl)-2-oxo-5-oxazolidinyl)methyl-trifluoroacetamide (Compound No. 54),
   their pharmaceutically acceptable salts, pharmaceutically acceptable solvates, enantiomers, diasteromers, polymorphs or N-oxides.

The present invention also encompasses pharmaceutical compositions comprising a therapeutically effective amount of a compound of Formula I together with a pharmaceutically acceptable carrier, excipient or diluent.

Compounds of the present invention can be useful antimicrobial agents. Accordingly, other embodiments of the invention are directed to methods of treating or preventing a condition caused by or contributed to by bacterial infection in a mammal in need thereof comprising administering to the mammal a therapeutically effective amount of a compound of Formula I or a pharmaceutical composition comprising a therapeutically effective amount of a compound of Formula I. In particular, the compounds of the present invention can treat, for example, community-acquired pneumonia, upper and lower respiratory tract infections, skin and soft tissue infections, bone and joint infections, hospital-acquired lung infections, mastitis, catheter infection and foreign body or prosthesis infections. The bacterial infection treated by these methods can be caused by or contributed to by one or more gram-positive or gram-negative bacterium, and in some cases, Staphylococci, Streptococci, Enterococci, Bacterioides spp., Clostridium spp., Mycobacterium spp., Bacillus spp., Corynebacterium spp., Peptostreptococcus spp., Listeria spp., Legionella spp., Haemophilus influenza, Moraxella, Eschericia faecalis and Eschericia coli. The methods are particularly useful in treating bacterial conditions caused by or contributed to by drug-resistant bacterium.

The present invention also encompasses a process for preparing a compound of Formula IV,

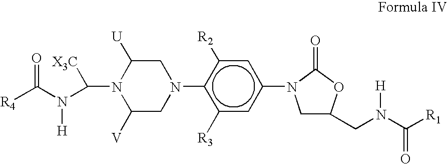

and its pharmaceutically acceptable salts, pharmaceutically acceptable solvates, enantiomers, diasteromers, N-oxides or polymorphs, wherein
R₁ and R₄ can be hydrogen, optionally substituted alkyl, alkenyl, alkynyl, or cycloalkyl, (wherein optional substituents are selected from one or more of halogen, hydroxy or alkoxy), and optionally substituted aryl or heterocycle (substituted with one or more of halogen, hydroxy, mercapto, alkoxy, alkyl, acyl, acyloxy, haloalkyl, amino, cyano, nitro, thio or thioalkyl);
R₂ and R₃ can be hydrogen, halogen, alkyl, or haloalkyl;
U and V can be hydrogen, alkyl, or haloalkyl; and
X can be halogen,

which method comprises reacting a compound of Formula II with a compound of Formula III

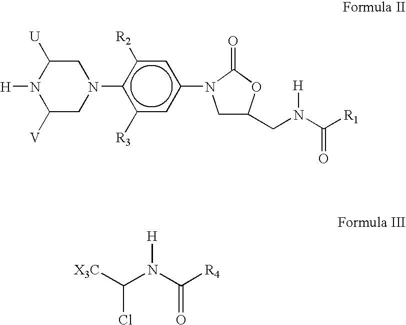

The present invention also encompasses a process for the preparation of compound of Formula VI,

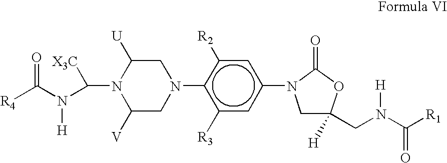

its pharmaceutically acceptable salts, pharmaceutically acceptable solvates, enantiomers, diasteromers, N-oxide or polymorphs, wherein
R₁ and R₄ can be hydrogen, optionally substituted alkyl, alkenyl, alkynyl, cycloalkyl (substituted with one or more of halogen, hydroxy or alkoxy) or optionally substituted aryl or heterocycle substituted with one or more of halogen, hydroxy, mercapto, alkoxy, alkyl, acyl, acyloxy, haloalkyl, amino, cyano, nitro, thio or thioalkyl;
R₂ and R₃ can be hydrogen, halogen, alkyl or haloalkyl;
U and V can be hydrogen, alkyl, or haloalkyl; and
X can be halogen,

which method comprises reacting a compound of Formula V with a compound of Formula III

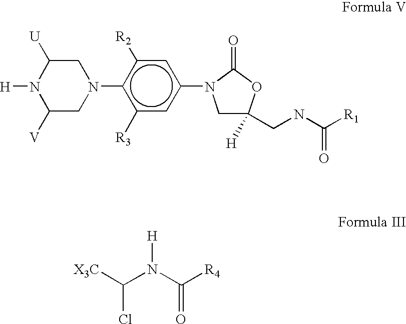

to give a compound of Formula VI.

The processes described above can be carried out in nonpolar solvents, such as, for example, dichloromethane, chloroform, carbon tetrachloride and dichloroethane. In addition, the processes described above can be carried out in the presence of an organic base, such as, for example, triethylamine, pyridine, diisopropylamine, propylamine and N-methylamine.

Unless otherwise defined, all technical and scientific terms used herein have the same ordinary meaning as commonly understood by one of ordinary skill in the art. Although methods and materials similar or equivalent to those described herein can be used in the practice or testing of the present invention, suitable methods and materials are described below. All publications, patent applications, patents and other references disclosed herein are incorporated by reference in their entirety. In case of conflict, the present specification, including all definitions, will control. Further, the materials, methods and examples are illustrative only and are not intended to be limiting.

The term “alkyl,” as used herein, refers to branched or unbranched saturated hydrocarbon chain having from 1 to 6 carbon atoms. Examples of alkyl groups include, but are not limited to, methyl, ethyl, n-propyl, iso-propyl, n-butyl, iso-butyl, t-butyl, n-hexyl and the like.

The term “alkenyl,” as used herein, refers to branched or unbranched unsaturated hydrocarbon chain having from 2 to 6 carbon atoms. Examples of alkenyl groups include, but are not limited to, ethylene, propylene and the like.

The term “alkynyl,” as used herein, refers to branched or unbranched unsaturated hydrocarbon chain having from 2 to 6 carbon atoms. Examples of alkynyl groups include, but are not limited to, ethynyl, propynyl and the like.

The term “cycloalkyl,” as used herein, refers to cyclic hydrocarbons having from 3 to 7 carbon atoms having a single cyclic ring or multiple condensed rings. Examples of cycloalkyl groups include, but are not limited to, cyclopropyl, cyclobutyl, cyclopentyl and the like.

The term “alkoxy,” as used herein, refers to the group R—O— wherein R is cycloalkyl or alkyl. Examples of alkoxy groups include, but are not limited to, methoxy, ethoxy, propoxy, isopropoxy, cyclopropyloxy, cyclohexyloxy and the like.

The term “thio,” as used herein, refers to the group —SH.

The term “thioalkyl,” as used herein, refers to —S-alkyl.

The term “haloalkyl” as used herein refers to alkyl having one or more hydrogen(s) replaced by halogen, such as F, Cl, Br or I.

The term “acyl,” as used herein, refers to —C(O)R wherein R is alkyl, cycloalkyl, heterocyclyl or aryl.

The term “acyloxy,” as used herein, refers to —OC(O)R wherein R is hydrogen, alkyl, cycloalkyl, heterocyclyl or aryl.

The term “halogen,” as used herein, refers to fluorine, chlorine, bromine or iodine.

The term “aryl,” as used herein, stands for an aromatic radical having 6 to 14 carbon atoms. Examples of aryl groups include, but are not limited to, phenyl, naphthyl, anthryl, biphenyl, and the like.

The term “heterocycle,” as used herein, refers to a non-aromatic or aromatic ring system having one or more heteroatom(s) wherein the heteroatom(s) is/are selected from the group consisting of nitrogen, sulfur and oxygen and the ring system refers to mono-, bi- or tricyclic systems. Examples of heterocycle groups include, but are not limited to, thienyl, furyl, pyrolyl, pyrazolyl, pyridyl, pyrimidyl, pyrazinyl, pyridazinyl, quinolinyl, isoquinolinyl, quinazolinyl, quinoxalinyl, phthalazinyl, cinnolinyl, thiazolyl, benzothiazolyl, isothiazolyl, oxazolyl, benzoxazolyl, isoxazolyl, imidazolyl, benzimidazolyl, pyrazolyl, indolyl, isoindolyl and the like.

The term “polymorphs,” as used herein, includes all crystalline forms for compounds described herein. In addition, some of the compounds described herein may form solvates with water (i.e., hydrate, hemihydrate or sesquihydrate) or organic solvents. Such solvates are also encompassed within the scope of this invention.

The term “drug-resistance,” as used herein, refers to the characteristics of a microbe to survive in the presence of a currently available antimicrobial agent at its routine, effective concentration.

The phrase “pharmaceutically acceptable salts,” as used herein, refers to salts of the free base, which substantially possess the desired pharmacological activity of the free base and which are neither biologically nor otherwise undesirable. Suitable pharmaceutically acceptable salts may be prepared from pharmaceutically acceptable non-toxic acids, but are not limited to inorganic acids, organic acids, solvates, hydrates or clathrates thereof. Example of such inorganic acids include, but not limited to, hydrochloric, hydrobromic, hydroiodic, nitrous (nitrite salt), nitric (nitrate salt), carbonic, sulfuric, phosphoric acid and like. Appropriate organic acids include, but are not limited to, aliphatic, cycloaliphatic, aromatic, heterocyclic, carboxylic and sulfonic classes of organic acids, such as, for example, formic, acetic, propionic, succinic, glycolic, gluconic, lactic, malic, tartaric, citric, ascorbic, glucuronic, maleic, fumaric, pyruvic, aspartic, glutamic, benzoic, anthranilic, mesylic, salicylic, p-hydroxybenzoic, phenylacetic, mandelic, embonic (pamoic), methanesulfonic, ethanesulfonic, benzenesulfonic, pantothenic, toluenesulfonic, 2-hydroxyethanesulfonic, sulfanilic, stearic, algenic, beta-hydroxybutyric, cyclohexylaminosulfonic, galactaric, galacturonic acid and the like.

Compounds disclosed herein contain one or more asymmetric carbon atoms and thus can exist as racemates and racemic mixtures, single enantiomers, diastereomeric mixtures and individual diastereomers. All such isomeric forms of these compounds are expressly included herein. Each stereogenic carbon may be of the R or S configuration. Although the specific compounds exemplified in this application may be depicted in a particular stereochemical configuration, compounds having either the opposite stereochemistry at any given chiral center or mixtures thereof are encompassed within the scope of the invention. Although amino acids and amino acid side chains may be depicted in a particular configuration, both natural and unnatural forms are encompassed within the scope of the invention.

### DETAILED DESCRIPTION OF THE INVENTION

The compounds disclosed herein may be prepared by the following reaction sequences as depicted in Schemes I and II defined below.

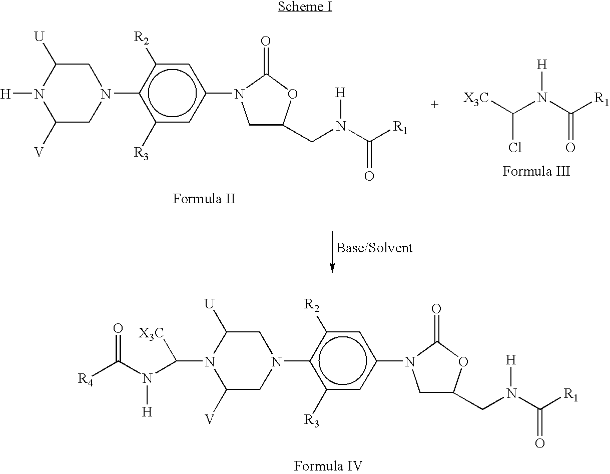

In Scheme I, compounds of Formula IV (wherein R₁ and R₄ can be hydrogen; optionally substituted alkyl, alkenyl, alkynyl, or cycloalkyl (substituted with one or more of halogen, hydroxyl or alkoxy); and optionally substituted aryl or heterocycle (substituted with one or more of halogen, hydroxy, mercapto, alkoxy, alkyl, acyl, acyloxy, haloalkyl, amino, cyano, nitro, thio, or thioalkyl); R₂ and R₃ can be hydrogen, halogen, alkyl, or haloalkyl; U and V can be hydrogen, alkyl, or haloalkyl; and X may be halogen) may be prepared by reacting a compound of Formula II (available according to procedures disclosed in any of U.S. Pat. No. 5,547,950; S. E. Scaus and E. N. Jacobson, Tetrahedron Letter, 37(44):7937 (1996); and K. C. Grega et al, J. Org. Chem., 60(16):5255 (1995)) with a compound of Formula III (available according to procedures disclosed in either of Z. Chem., 27(2):70 (1987); or Coll. Czech. Chem. Commu., 50:1019 (1995)). In particular, a compound of Formula II reacts with a compound of Formula III in a solvent and in the presence of an organic base to yield a compound of Formula IV. Preferably, the solvent is a nonpolar solvent, including, but not limited to, chloroform, dichloromethane, carbon tetrachloride or dicholoroethane. Preferably, the organic base includes, but is not limited to, triethylamine, pyridine, diisopropylamine, propylamine, N-methylamine and the like.

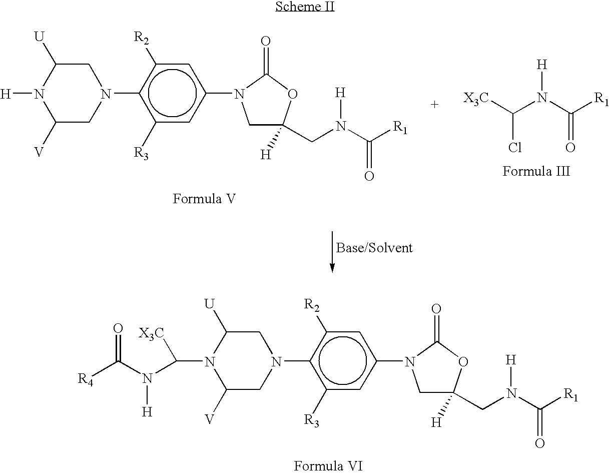

The compound of Formula VI (wherein R₁ and R₄ can be hydrogen; optionally substituted alkyl, alkenyl, alkynyl, or cycloalkyl (substituted with one or more of halogen, hydroxyl or alkoxy); and optionally substituted aryl or heterocycle (substituted with one or more of halogen, hydroxy, mercapto, alkoxy, alkyl, acyl, acyloxy, haloalkyl, amino, cyano, nitro, thio, or thioalkyl); R₂ and R₃ can be hydrogen, halogen, alkyl, or haloalkyl; U and V can be hydrogen, alkyl, or haloalkyl; and X can be halogen) can be prepared according to Scheme II. In particular, reacting a compound of Formula V (available according to procedures disclosed in S. J. Bricker et al., J. Med. Chem., 39:673 (1996)) with a compound of Formula III (available according to procedures disclosed in either of Z. Chem., 27(2):70 (1987); or Coll. Czech. Chem. Commu., 50:1019 (1995)). In particular, a compound of Formula V reacts with a compound of Formula III in a solvent and in the presence of an organic base to yield a compound of Formula VI. Preferably, the solvent is a nonpolar solvent, including, but not limited to, chloroform, dichloromethane, carbon tetrachloride or dicholoroethane. Preferably, the organic base includes, but is not limited to, triethylamine, pyridine, diisopropylamine, propylamine, N-methylamine and the like.

In the above schemes, where the specific bases, solvents, etc., are mentioned, it is understood that bases, solvents, etc., known to one of ordinary skill in the art also may be used. Similarly, the reaction temperature and duration may be adjusted as desired.

The compounds disclosed herein can possess significant antimicrobial activity against gram-positive and certain gram-negative bacteria. Such compounds are useful as antibacterial agents for the treatment or prevention of bacterial infections in humans and animals. Specific compounds according to the invention include the following (also shown in Table I).
N-((3-(4-(3-Fluoro-4-(1′-formamido-2′,2′,2′-trichloroethyl)-1-piperazinyl)phenyl)-2-oxo-5-oxazolidinyl)methyl)-acetamide (Compound No. 1)
N-((3-(4-(3-Fluoro-4-(1′-acetamido-2′,2′,2′-trichloroethyl)-1-piperazinyl)phenyl)-2-oxo-5-oxazolidinyl)methyl)-acetamide (Compound No. 2)
N-((3-(4-(3-Fluoro-4-(1′-trichloroacetamido-2′,2′,2′-trichloroethyl)-1-piperazinyl)phenyl)-2-oxo-5-oxazolidinyl)methyl)-acetamide (Compound No. 3)
N-((3-(4-(3-Fluoro-4-(1′-trifluoroacetamido-2′,2′,2-trichloroethyl)-1-piperazinyl)phenyl)-2-oxo-5-oxazolidinyl)methyl)-acetamide (Compound No. 4)
N-((3-(4-(3-Fluoro-4-(1′-formamido-2′,2′,2′-tribromoethyl)-1-piperazinyl)phenyl)-2-oxo-5-oxazolidinyl)methyl)-acetamide (Compound No. 5)
N-((3-(4-(3-Fluoro-4-(1′-acetamido-2′,2′,2′-tribromoethyl)-1-piperazinyl)phenyl)-2-oxo-5-oxazolidinyl)methyl)-acetamide (Compound No. 6)
N-((3-(4-(3-Fluoro-4-(1′-trifluoroacetamido-2′,2′,2′-tribromoethyl)-1-piperazinyl)phenyl)-2-oxo-5-oxazolidinyl)methyl)-acetamide (Compound No. 7)
N-((3-(4-(3-Fluoro-4-(1′-benzamido-2′,2′,2′-trichloroethyl)-1-piperazinyl)phenyl)-2-oxo-5-oxazolidinyl)methyl)-acetamide (Compound No. 8)
N-((3-(4-(3-Fluoro-4-(1′-(2,4-dichloro-5-fluorobenzamido)-2′,2′,2′-trichloroethyl)-1-piperazinyl)phenyl)-2-oxo-5-oxazolidinyl)methyl)-acetamide (Compound No. 9)
N-((3-(4-(3-Fluoro-4-(1′-benzamido-2′,2′,2′-tribromoethyl)-1-piperazinyl)phenyl)-2-oxo-5-oxazolidinyl)methyl)-acetamide (Compound No. 10)
N-((3-(4-(3-Fluoro-4-(1′-(2,4-dichloro-5-fluorobenzamido)-2′,2′,2′-tribromoethyl)-1-piperazinyl)phenyl)-2-oxo-5-oxazolidinyl)methyl)-acetamide (Compound No. 11)
N-((3-(4-(3-Fluoro-4-(1′-formamido-2′,2′,2′-trifluoroethyl)-1-piperazinyl)phenyl)-2-oxo-5-oxazolidinyl)methyl)-acetamide (Compound No. 12)
(S)—N-((3-(4-(3-Fluoro-4-(1′-formamido-2′,2′,2′-trichloroethyl)-1-piperazinyl)phenyl)-2-oxo-5-oxazolidinyl)methyl)-acetamide (Compound No. 13)
(S)—N-((3-(4-(3-Fluoro-4-(1′-acetamido-2′,2′,2′-trichloroethyl)-1-piperazinyl)phenyl)-2-oxo-5-oxazolidinyl)methyl)-acetamide (Compound No. 14)
(S)—N-((3-(4-(3-Fluoro-4-(1′-trichloroacetamido-2′,2′,2′-trichloroethyl)-1-piperazinyl)phenyl)-2-oxo-5-oxazolidinyl)methyl)-acetamide (Compound No. 15)
(S)—N-(3-(4-(3-Fluoro-4-(1′-trifluoroacetamido-2′,2′,2′-trichloroethyl)-1-piperazinyl)phenyl)-2-oxo-5-oxazolidinyl)methyl)-acetamide (Compound No. 16)
(S)—N-((3-(4-(3-Fluoro-4-(1′-formamido-2′,2′,2′-tribromoethyl)-1-piperazinyl)phenyl)-2-oxo-5-oxazolidinyl)methyl)-acetamide (Compound No. 17)
(S)—N-((3-(4-(3-Fluoro-4-(1′-acetamido-2′,2′,2′-tribromoethyl)-1-piperazinyl)phenyl)-2-oxo-5-oxazolidinyl)methyl)-acetamide (Compound No. 18)
(S)—N-((3-(4-(3-Fluoro-4-(1′-trichloroacetamido-2′,2′,2′-tribromoethyl)-1-piperazinyl)phenyl)-2-oxo-5-oxazolidinyl)methyl)-acetamide (Compound No. 19)
(S)—N-((3-(4-(3-Fluoro-4-(1′-trifluoroacetamido-2′,2′,2′-tribromoethyl)-1-piperazinyl)phenyl)-2-oxo-5-oxazolidinyl)methyl)-acetamide (Compound No. 20)
(S)—N-((3-(4-(3-Fluoro-4-(1′-formamido-2′,2′,2′-trifluoroethyl)-1-piperazinyl)phenyl)-2-oxo-5-oxazolidinyl)methyl)-acetamide (Compound No. 21)
(S)—N-((3-(4-(3-Fluoro-4-(1′-acetamido-2′,2′,2′-trifluoroethyl)-1-piperazinyl)phenyl)-2-oxo-5-oxazolidinyl)methyl)-acetamide (Compound No. 22)
(S)—N-((3-(4-(3-Fluoro-4-(1′-formamido-2′,2′,2′-trichloroethyl)-1-piperazinyl)phenyl)-2-oxo-5-oxazolidinyl)methyl)-trifluoroacetamide (Compound No. 23)
(S)—N-((3-(4-(3-Fluoro-4-(1′-acetamido-2′,2′,2′-trichloroethyl)-1-piperazinyl)phenyl)-2-oxo-5-oxazolidinyl)methyl)-trifluoroacetamide (Compound No. 24)
(S)—N-((3-(4-(3-Fluoro-4-(1′-trichloroacetamido-2′,2′,2′-trichloroethyl)-1-piperazinyl)phenyl)-2-oxo-5-oxazolidinyl)methyl)-trifluoroacetamide (Compound No. 25)
(S)—N-((3-(4-(3-Fluoro-4-(1′-trifluoroacetamido-2′,2′,2′-trichloroethyl)-1-piperazinyl)phenyl)-2-oxo-5-oxazolidinyl)methyl)-trifluoroacetamide (Compound No. 26)
(S)—N-((3-(4-(3-Fluoro-4-(1′-formamido-2′,2′,2′-tribromoethyl)-1-piperazinyl)phenyl)-2-oxo-5-oxazolidinyl)methyl)-trifluoroacetamide (Compound No. 27)
(S)—N-((3-(4-(3-Fluoro-4-(1′-acetamido-2′,2′,2′-tribromoethyl)-1-piperazinyl)phenyl)-2-oxo-5-oxazolidinyl)methyl)-trifluoroacetamide (Compound No. 28)
(S)—N-((3-(4-(3-Fluoro-4-(1′-trichloroacetamido-2′,2′,2′-tribromoethyl)-1-piperazinyl)phenyl)-2-oxo-5-oxazolidinyl)methyl)-trifluoroacetamide (Compound No. 29)
(S)—N-((3-(4-(3-Fluoro-4-(1′-trifluoroacetamido-2′,2′,2′-tribromoethyl)-1-piperazinyl)phenyl)-2-oxo-5-oxazolidinyl)methyl)-trifluoroacetamide (Compound No. 30)
(S)—N-((3-(4-(3-Fluoro-4-(1′-formamido-2′,2′,2′-trifluoroethyl)-1-piperazinyl)phenyl)-2-oxo-5-oxazolidinyl)methyl)-trifluoroacetamide (Compound No. 31)
(S)—N-((3-(4-(3-Fluoro-4-(1′-acetamido-2′,2′,2′-trifluoroethyl)-1-piperazinyl)phenyl)-2-oxo-5-oxazolidinyl)methyl)-trifluoroacetamide (Compound No. 32)
(S)—N-((3-(4-(3-Fluoro-4-(1′-trichloroacetamido-2′,2′,2′-trifluoroethyl)-1-piperazinyl)phenyl)-2-oxo-5-oxazolidinyl)methyl)-trifluoroacetamide (Compound No. 33)
(S)—N-((3-(4-(3-Fluoro-4-(1′-formamido-2′,2′,2′-trichloroethyl)-3-methyl-1-piperazinyl)phenyl)-2-oxo-5-oxazolidinyl)methyl)-acetamide (Compound No. 34)
(S)—N-((3-(4-(3-Fluoro-4-(1′-acetamido-2′,2′,2′-trichloroethyl)-3-methyl-1-piperazinyl)phenyl)-2-oxo-5-oxazolidinyl)methyl)-acetamide (Compound No. 35)
(S)—N-((3-(4-(3-Fluoro-4-(1′-trichloroacetamido-2′,2′,2′-trichloroethyl)-3-methyl-1-piperazinyl)phenyl)-2-oxo-5-oxazolidinyl)methyl)-acetamide (Compound No. 36)
(S)—N-((3-(4-(3-Fluoro-4-(1′-trifluoroacetamido-2′,2′,2′-trichloroethyl)-3-methyl-1-piperazinyl)phenyl)-2-oxo-5-oxazolidinyl)methyl)-acetamide (Compound No. 37)
(S)—N-((3-(4-(3-Fluoro-4-(1′-formamido-2′,2′,2′-tribromoethyl)-3-methyl-1-piperazinyl)phenyl)-2-oxo-5-oxazolidinyl)methyl)-acetamide (Compound No. 38)
(S)—N-((3-(4-(3-Fluoro-4-(1′-acetamido-2′,2′,2′-tribromoethyl)-3-methyl-1-piperazinyl)phenyl)-2-oxo-5-oxazolidinyl)methyl)-acetamide (Compound No. 39)
(S)—N-((3-(4-(3-Fluoro-4-(1′-trichloroacetamido-2′,2′,2′-tribromoethyl)-3-methyl-1-piperazinyl)phenyl)-2-oxo-5-oxazolidinyl)methyl)-acetamide (Compound No. 40)
(S)—N-((3-(4-(3-Fluoro-4-(1′-trifluoroacetamido-2′,2′,2′-tribromoethyl)-3-methyl-1-piperazinyl)phenyl)-2-oxo-5-oxazolidinyl)methyl)-acetamide (Compound No. 41)
(S)—N-((3-(4-(3-Fluoro-4-(1′-formamido-2′,2′,2′-trifluoroethyl)-3-methyl-1-piperazinyl)phenyl)-2-oxo-5-oxazolidinyl)methyl)-acetamide (Compound No. 42)
(S)—N-((3-(4-(3-Fluoro-4-(1′-acetamido-2′,2′,2′-trifluorethyl)-3-methyl-1-piperazinyl)phenyl)-2-oxo-5-oxazolidinyl)methyl)-acetamide (Compound No. 43)
(S)—N-((3-(4-(3-Fluoro-4-(1′-trichloroacetamido-2′,2′,2′-trifluoroethyl)-3-methyl-1-piperazinyl)phenyl)-2-oxo-5-oxazolidinyl)methyl)-acetamide (Compound No. 44)
(S)—N-((3-(4-(3-Fluoro-4-(1′-formamaido-2′,2′,2′-tricholroethyl)-3-methyl-1-piperazinyl)phenyl)-2-oxo-5-oxazolidinyl)methyl)-trifluoroacetamide (Compound No. 45)
(S)—N-((3-(4-(3-Fluoro-4-(1′-acetamido-2′,2′,2′-trichloroethyl)-3-methyl-1-piperazinyl)phenyl)-2-oxo-5-oxazolidinyl)methyl)-trifluoroacetamide (Compound No. 46)
(S)—N-((3-(4-(3-Fluoro-4-(1′-trichloroacetamido-2′,2′,2′-trichloroethyl)-3-methyl-1-piperazinyl)phenyl)-2-oxo-5-oxazolidinyl)methyl)-trifluoroacetamide (Compound No. 47)
(S)—N-((3-(4-(3-Fluoro-4-(1′-trifluoroacetamido-2′,2′,2′-trichloroethyl)-3-methyl-1-piperazinyl)phenyl)-2-oxo-5-oxazolidinyl)methyl)-trifluoroacetamide (Compound No. 48)
(S)—N-((3-(4-(3-Fluoro-4-(1′-formamido-2′,2′,2′-tribromoethyl)-3-methyl-1-piperazinyl)phenyl)-2-oxo-5-oxazolidinyl)methyl)-trifluoroacetamide (Compound No. 49)
(S)—N-((3-(4-(3-Fluoro-4-(1′-acetamido-2′,2′,2′-tribromoethyl)-3-methyl-1-piperazinyl)phenyl)-2-oxo-5-oxazolidinyl)methyl)-trifluoroacetamide (Compound No. 50)
(S)—N-((3-(4-(3-Fluoro-4-(1′-trichloroacetamido-2′,2′,2′-tribromoethyl)-3-methyl-1-piperazinyl)phenyl)-2-oxo-5-oxazolidinyl)methyl)-trifluoroacetamide (Compound No. 51)
(S)—N-((3-(4-(3-Fluoro-4-(1′-trifluoroacetamido-2′,2′,2′-tribromoethyl)-3-methyl-1-piperazinyl)phenyl)-2-oxo-5-oxazolidinyl)methyl)-trifluoroacetamide (Compound No. 52)
(S)—N-((3-(4-(3-Fluoro-4-(1′-formamido-2′,2′,2′-trifluoroethyl)-3-methyl-1-piperazinyl)phenyl)-2-oxo-5-oxazolidinyl)methyl)-trifluoroacetamide (Compound No. 53)
(S)—N-((3-(4-(3-Fluoro-4-(1′-acetamido-2′,2′,2′-trifluoroethyl)-3-methyl-1-piperazinyl)phenyl)-2-oxo-5-oxazolidinyl)methyl)-trifluoroacetamide (Compound No. 54)

TABLE I

| | | | | | | Formula I |
|---|---|---|---|---|---|---|
| 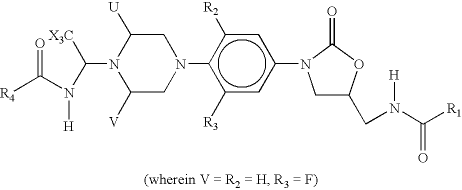 | | | | | | |
| Compound No. | R₁ | U | X | R₄ | [α]^{D} | m.p. ° C. |
| 1 | —CH₃ | H | Cl | H | RM | 186 |
| 2 | —CH₃ | H | Cl | —CH₃ | RM | 205 |
| 3 | —CH₃ | H | Cl | —CCl₃ | RM | 179 |
| 4 | —CH₃ | H | Cl | —CF₃ | RM | 210 |
| 5 | —CH₃ | H | Br | H | RM | 197 |
| 6 | —CH₃ | H | Br | —CH₃ | RM | Liquid |
| 7 | —CH₃ | H | Br | —CF₃ | RM | 202 |
| 8 | —CH₃ | H | Cl | —C₆H₅ | RM | 175-176 |
| 9 | —CH₃ | H | Cl | 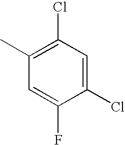 | RM | Liquid |
| 10 | —CH₃ | H | Br | —C₆H₅ | RM | 245 |
| 11 | —CH₃ | H | Br | 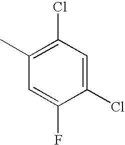 | RM | Liquid |
| 12 | —CH₃ | H | F | H | RM | 185 |
| 13 | —CH₃ | H | Cl | H | −12.27 | 107 |
| | | | | | (MeOH) | |
| 14 | —CH₃ | H | Cl | —CH₃ | −6.107 | 166 |
| | | | | | (CHCl₃) | |
| 15 | —CH₃ | H | Cl | —CCl₃ | −12.0 | 192 |
| | | | | | (CHCl₃) | |
| 16 | —CH₃ | H | Cl | —CF₃ | −10.11 | 198 |
| | | | | | (CHCl₃) | |
| 17 | —CH₃ | H | Br | H | −10.17 | 128 |
| | | | | | (MeOH) | |
| 18 | —CH₃ | H | Br | —CH₃ | −7.76 | 139 |
| | | | | | (acetone) | |
| 19 | —CH₃ | H | Br | —CCl₃ | −19.80 | 181 |
| | | | | | (MeOH) | |
| 20 | —CH₃ | H | Br | —CF₃ | −11.88 | 192 |
| | | | | | (CHCl₃) | |
| 21 | —CH₃ | H | F | H | −17.67 | 170 |
| | | | | | (MeOH) | |
| 22 | —CH₃ | H | F | —CH₃ | −15.36 | 192 |
| | | | | | (MeOH) | |
| 23 | —CF₃ | H | Cl | H | −31.51 | 172 |
| | | | | | (MeOH) | |
| 24 | —CF₃ | H | Cl | —CH₃ | −12.7 | 174 |
| | | | | | (MeOH) | |
| 25 | —CF₃ | H | Cl | —CCl₃ | −33.64 | 173 |
| | | | | | (MeOH) | |
| 26 | —CF₃ | H | Cl | —CF₃ | −34.92 | 170 |
| | | | | | (MeOH) | |
| 27 | —CF₃ | H | Br | H | −7.18 | 143 |
| | | | | | (MeOH) | |
| 28 | —CF₃ | H | Br | —CH₃ | −18.99 | 149 |
| | | | | | (MeOH) | |
| 29 | —CF₃ | H | Br | —CCl₃ | NA | 221 |
| 30 | —CF₃ | H | F | —CF₃ | −27.0 | 160 |
| | | | | | (MeOH) | |
| 31 | —CF₃ | H | F | H | NA | 171 |
| 32 | —CF₃ | H | F | —CH₃ | −26.9 | 208 |
| | | | | | (MeOH) | |
| 33 | —CF₃ | H | F | —CCl₃ | −21.54 | 60 |
| | | | | | (MeOH) | |
| 34 | —CH₃ | —CH₃ | Cl | H | −11.18 | Syrup |
| | | | | | (MeOH) | |
| 35 | —CH₃ | —CH₃ | Cl | —CH₃ | −13.60 | Syrup |
| | | | | | (MeOH) | |
| 36 | —CH₃ | —CH₃ | Cl | —CCl₃ | −16.49 | Syrup |
| | | | | | (MeOH) | |
| 37 | —CH₃ | —CH₃ | Cl | —CF₃ | −16.32 | 87 |
| | | | | | (MeOH) | |
| 38 | —CH₃ | —CH₃ | Br | H | −7.02 | Syrup |
| | | | | | (MeOH) | |
| 39 | —CH₃ | —CH₃ | Br | —CH₃ | −11.66 | Syrup |
| | | | | | (MeOH) | |
| 40 | —CH₃ | —CH₃ | Br | —CCl₃ | −5.97 | Syrup |
| | | | | | (MeOH) | |
| 41 | —CH₃ | —CH₃ | Br | —CF₃ | −15.13 | Syrup |
| | | | | | (MeOH) | |
| 42 | —CH₃ | —CH₃ | F | H | −11.66 | Syrup |
| | | | | | (MeOH) | |
| 43 | —CH₃ | —CH₃ | F | —CH₃ | −11.13 | Syrup |
| | | | | | (MeOH) | |
| 44 | —CH₃ | —CH₃ | F | —CCl₃ | −9.19 | 96 |
| | | | | | (MeOH) | |
| 45 | —CF₃ | —CH₃ | Cl | H | −18.30 | 108 |
| | | | | | (MeOH) | |
| 46 | —CF₃ | —CH₃ | Cl | —CH₃ | −19.70 | 116 |
| | | | | | (MeOH) | |
| 47 | —CF₃ | —CH₃ | Cl | —CCl₃ | −32.77 | 88 |
| | | | | | (MeOH) | |
| 48 | —CF₃ | —CH₃ | Cl | —CF₃ | −28.52 | 77 |
| | | | | | (MeOH) | |
| 49 | —CF₃ | —CH₃ | Br | H | −13.61 | Syrup |
| | | | | | (MeOH) | |
| 50 | —CF₃ | —CH₃ | Br | —CH₃ | −15.92 | Syrup |
| | | | | | (MeOH) | |
| 51 | —CF₃ | —CH₃ | Br | —CCl₃ | −23.88 | Syrup |
| | | | | | (MeOH) | |
| 52 | —CF₃ | —CH₃ | Br | —CF₃ | −27.65 | 110 |
| | | | | | (MeOH) | |
| 53 | —CF₃ | —CH₃ | F | H | −21.43 | 92 |
| | | | | | (MeOH) | |
| 54 | —CF₃ | —CH₃ | F | —CH₃ | −22.44 | 140 |
| | | | | | (MeOH) | |

RM: Racemic mixture
NO: not observed

This invention further provides methods of treating or preventing bacterial infections, or enhancing or potentiating the activity of other antibacterial agents, in a mammal having conditions caused by or contributed to by bacterial infection, which comprises administering to the mammal a compound of the invention alone or in admixture in the form of a medicament according to the invention. The terms “treating” or “treatment” include administering, either simultaneously, separately or sequentially, a therapeutically effective amount of a composition containing one or more of the compounds disclosed herein to a mammal that desires inhibition of bacterial growth.

The phrase “therapeutically effective amount” as used herein refers to that amount of a therapeutic agent, which provides a therapeutic benefit in the treatment or prevention of a condition caused by or contributed to by bacterial infection. The therapeutically effective amount of the compound for treatment varies depending on the manner of administration, the age, weight, and general health of the mammal treated, and ultimately will be decided by the physicians.

Compounds disclosed herein display antimicrobial activity against multiply-resistant microorganisms, such as Staphylococcus aureus, Staphylococcus epidermidis, Streptococcus pneumonia, S. pyogen, S. bovis, S. equi, S. mutans, M. catarrhalis, Enterococcus faecalis, Enterococcus faecium, Enterococcus durans, Eschericia coli, Salmonella, K. oxytosa, P. aeruginosa, S. marcescens, Acinetobacter, and the like. These compounds also can be useful in the treatment of community-acquired pneumonia, upper and lower respiratory tract infections, skin and soft tissue infections, hospital-acquired lung infections, bone and joint infections, and other bacterial infections, such as mastitis, catheter infection, foreign body or prosthesis infections.

The compounds of the present invention may be administered to a mammal, such as human, by any route appropriate to the condition to be treated, suitable routes including oral, rectal, nasal, topical (including buccal and sublingual), vaginal and parenteral (including subcutaneous, intramascular, intravenous, intradermal, intrathecal and epidural). The preferred route may vary with, for example, the condition of the recipient, as well the ease of preparation and administration.

The pharmaceutical compositions of the present invention comprise a therapeutically effective amount of a compound described herein formulated together with one or more pharmaceutically acceptable carriers, excipients or diluents. The term “pharmaceutically acceptable carriers” is intended to include non-toxic, inert solid, semi-solid or liquid filler, encapsulating material or formulation auxiliary of any type.

The pharmaceutical compositions of the present invention include solid compositions for oral administrations, which include capsules, tablet, pills, powder, granules, sachets and suppository. The solid compositions typically are formed by mixing the active compound with at least one inert, pharmaceutically acceptable carrier, excipient or diluent, such as but not limited to sodium citrate, dicalcium phosphate and/or a filler or extenders, such as but not limited to starches, lactose, sucrose, glucose, mannitol and silicic acid; binders, such as but not limited to carboxymethylcellulose, alginates, gelatins, polyvinylpyrrolidinone, sucrose, acacia; disintegrating agents, such as but not limited to agar-agar, calcium carbonate, potato starch, alginic acid, certain silicates and sodium carbonate; absorption accelerators, such as but not limited to quaternary ammonium compounds; wetting agents, such as but not limited to cetyl alcohol, glycerol monostearate; adsorbants, such as but not limited to Kaolin and the like; lubricants, such as but not limited to talc, calcium stearate, magnesium stearate, solid polyethylene glycol, sodium lauryl sulfate and mixture thereof. In the case of capsules, tablets and pills, the dosage form may also comprise buffering agents.

Solid compositions, including tablets, capsules, pills granules and the like, can be prepared using coating and shells, such as enteric coating and other coatings well known in the pharmaceutical formulating art.

Liquid form compositions for oral administration include pharmaceutically acceptable emulsions, solutions, suspensions, syrups and elixirs. For liquid form compositions, the active compound is mixed with water or other solvent, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (such as cottonseed, groundnut, corn, germ, olive, castor and sesame oil), glycerol, and fatty acid esters of sorbitan and mixture thereof. In addition to inert diluents, the oral composition can also include adjuvants, such as wetting agents, emulsifying agents, suspending agents, sweetening agents, flavoring agents and perfuming agents.

Injectable compositions, such as sterile injections, aqueous suspensions and the like, may be formulated according to the art using suitable dispersing or wetting and suspending agent. Among the pharmaceutically acceptable vehicles and solvents that may be employed are water, Ringer's solution and isotonic sodium chloride.

Dosage forms for topical administration include ointments, pastes, creams, lotions, gels, powders, solutions, sprays, inhalants or patches. Typically, the active compound is admixed under sterile condition with a pharmaceutically acceptable carrier and any preservatives or buffers, as may be required. Ophthalmic formulations, eardrops, eye ointments, powders and solutions are also contemplated as being within the scope of this invention.

The pharmaceutical compositions of the present invention typically are in unit dosage form. In unit dosage form, the pharmaceutical compositions are subdivided into unit doses containing appropriate quantities of the active component. The unit dosage form can be a packaged preparation, the package containing discrete capsules, powders, in vials or ampoules, and ointments capsule, sachet, tablet, gel, cream itself or it can be the appropriate number of any of these packaged forms.

The quantity of active compound in unit dose of preparation may be varied or adjusted from less than 1 mg to several grams according to the particular application and potency of the active ingredient.

In therapeutic use for treating bacterial infections, the methods of the present invention utilize active compounds that can be administered at an initial dosage of about 3 mg to about 40 mg per kilogram daily. The dosages, however, may be varied depending upon the requirements of the patients and the compound being employed. Determination of the proper dosage for a particular situation is within the smaller dosages, which are less than the optimum dose. Small increments until the optimum effect under the daily dosage may be divided and administered in portion during the day if desired. It may be necessary to use dosages of each active ingredient outside the ranges disclosed herein in some cases, as will be readily apparent to those of ordinary skill in the art. Further, it is noted that the clinician or treating physician will know how and when to interrupt, adjust, or terminate therapy in conjunction with individual patient response.

Examples set forth below demonstrate the general synthetic procedure for the preparation of representative compounds. The examples are provided to illustrate particular aspects of the disclosure and do not constrain the scope of the present invention as defined by the claims. It will be apparent to those skilled in the art that many modifications, both to materials and methods, may be practiced without departing from the purpose and interest of this invention.

### Examples

### General Procedure

### Preparation of Compound of Formula Iv

A suspension of racemic compound of Formula II (1 equiv) in a solvent (2-5 mL) was cooled in an ice bath. A base (1.2 equiv) was added to this cooled suspension and stirred continuously. Compound of Formula III was added dropwise to this solution and stirred continuously. The mixture was diluted with chloroform and the solution was washed with water and brine. The chloroform layer was dried over anhydrous sodium sulfate and concentrated to afford the crude product. The crude product was purified by column chromatography using petroleum ether-ethylacetate mixture to afford the desired product.

The following compounds were prepared by following the above general procedure:

### Compound No. 1: N-((3-(4-(3-Fluoro-4-(1′-formamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 8.36 (s, 1H), 7.41 (dd, J=14.1 Hz, J=2.6 Hz, 1H), 6.90-7.20 (m, 3H), 5.39 (s, 1H), 4.78 (bm, 1H), 3.50-3.80 (m, 4H), 2.85-3.30 (m, 4H), 2.01 (s, 3H)

### Compound No. 2: N-((3-(4-(3-Fluoro-4-(1′-acetamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 7.38 (dd, J=14.1 Hz, J=2.6 Hz, 1H), 7.05 (d, J=8 Hz, 1H), 6.90 (t, J=9.1 Hz, 1H), 6.30-6.60 (m, 2H), 5.36 (s, 1H), 4.76 (bm, 1H), 4.01 (t, J=9 Hz, 1H), 3.55-3.80 (m, 4H), 2.80-3.20 (m, 8H), 2.17 (s, 1H), 2.02 (s, 3H)

### Compound No. 3: N-((3-(4-(3-Fluoro-4-(1′-trichloroacetamido-2′,2′,2′-trichloroethyl)-1-piperazinyl)phenyl)-2-oxo-5-oxazolidinyl)methyl)-acetamide

The following spectral identification confirmed product formation ¹HNMR (200 MHz, CDCl₃): δ 7.43 (dd, J=14.1 Hz, J=6.2 Hz, 1H), 7.05 (d, J=8 Hz, 1H), 6.92 (t, J=9.1 Hz, 1H), 6.24 (bt, J=11 Hz, 1H), 5.29 (d, 1H), 4.78 (bt, 1H), 4.02 (t, J=9 Hz, 1H), 3.55-3.80 (m, 4H), 2.90-3.30 (m, 8H), 2.02 (s, 3H)

### Compound No. 4: N-((3-(4-(3-Fluoro-4-(1′-trifluoroacetamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 8.10 (s, 1H), 7.47 (dd, J=14 Hz, J=2.6 Hz, 1H), 7.07 (d, J=8 Hz, 1H), 6.92 (t, J=9.1 Hz 1H), 6.20 (bt, J=11 Hz, 1H), 4.78 (bt, 1H), 4.03 (t, J=9 Hz, 1H), 3.50-3.80 (m, 8H), 2.95-3.15 (m, 4H), 2.03 (s, 3H)

### Compound No. 5: N-((3-(4-(3-Fluoro-4-(1′-formamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 8.10 (s, 1H), 7.47 (dd, J=14 Hz, J=2.6 Hz, 1H), 7.27 (m, 1H), 7.08 (d, J=8 Hz 1H), 6.92 (t, J=9.1 Hz, 1H), 6.10 (bt, J=11 Hz, 1H), 4.78 (bt, 1H), 4.03 (t, J=9 Hz, 1H), 3.50-3.80 (m, 8H), 2.95-3.15 (m, 4H), 2.03 (s, 3H)

### Compound No. 6: N-((3-(4-(3-Fluoro-4-(1′-acetamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 7.42 (dd, J=14.1 Hz, J=6.2 Hz, 1H), 7.07 (d, J=8 Hz, 1H), 6.92 (t, J=9.1 Hz, 1H), 6.18 (bt, J=11 Hz, 1H), 5.31 (d, 1H), 4.75 (bt, 1H), 4.02 (t, J=9 Hz, 1H), 2.85-3.80 (m, 12H), 2.18 (s, 3H), 2.03 (s, 3H)

### Compound No. 7: N-((3-(4-(3-Fluoro-4-(1′-trifluoroacetamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 8.10 (s, 1H), 7.45 (dd, J=14 Hz, J=2.6 Hz, 1H), 7.80 (d, J=8 Hz 1H), 6.92 (t, J=9.1 Hz 1H), 6.69 (bt, J=11 Hz, 1H), 4.79 (bt, 1H), 4.03 (t, J=9 Hz, 1H), 3.50-3.80 (m, 8H), 2.95-3.15 (m, 4H), 2.03 (s, 3H)

### Compound No. 8: N-((3-(4-(3-Fluoro-4-(1′-benzamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 7.83 (t, J=9 Hz, 2H), 7.35-7.60 (m, 4H), 7.02 (d, J=8 Hz, 1H), 6.91 (t, J=9.1 Hz, 1H), 6.81 (d, J=9 Hz, 1H), 6.54 (bt, J=11 Hz, 1H), 5.60 (d, 1H), 4.75 (bt, 1H), 4.0 (t, J=9 Hz, 1H), 3.60-3.80 (m, 4H), 2.85-3.80 (m, 8H), 2.01 (s, 3H)

### Compound No. 9: N-((3-(4-(3-Fluoro-4-(1′-(2,4-dichloro-5-fluorobenzamido)-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 7.35-7.70 (m, 4H), 7.02 (d, J=8 Hz, 1H), 6.90 (t, J=9.1 Hz, 1H), 6.70 (bt, J=11 Hz, 1H), 5.55 (d, 1H), 4.75 (bt, 1H), 4.00 (t, J=9 Hz, 1H), 3.60-3.80 (m, 4H), 2.90-3.30 (m, 8H), 2.00 (s, 3H)

### Compound No. 10: N-((3-(4-(3-Fluoro-4-(1′-benzamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 7.80 (d, 2H), 7.35-7.70 (m, 6H), 6.85-7.10 (m, 3H), 4.78 (bt, 1H), 4.0 (t, J=9 Hz, 1H), 3.0-3.80 (m, 12H), 2.01 (s, 3H)

### Compound No. 11: N-((3-(4-(3-Fluoro-4-(1′-(2,4-dichloro-5-fluorobenzamido)-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 7.71 (d, 2H), 7.51 (d, 1H), 7.27-7.45 (m, 3H), 7.03 (d, J=8 Hz, 1H), 6.92 (t, J=9.1 Hz, 1H), 6.39 (t, 1H), 4.78 (bt, 1H), 4.0 (t, J=9 Hz, 1H), 3.55-3.80 (m, 4H), 2.95-3.45 (m, 8H), 2.01 (s, 3H)

### Compound No. 12: N-((3-(4-(3-Fluoro-4-(1′-formamido-2′,2′,2′-trifluoroethyl)-1-piperazinyl)phenyl)-2-oxo-5-oxazolidinyl)methyl)-acetamide

The following spectral identification confirmed product formation. ¹HNMR (200 MHz, CDCl₃): δ 8.44 (bt, 1H), 8.25-8.35 (m, 1H), 7.45-7.70 (m, 2H), 6.90-7.15 (m, 2H), 5.47 (bm, 1H), 4.84 (bt, 1H), 4.11 (t, 1H), 3.60-3.90 (m, 4H), 2.80-3.20 (m, 8H), 2.02 (s, 3H)

### Preparation of S Isomer of Compound of Formula VI

A suspension of chiral compound of Formula V (1 equiv) in a solvent (2-5 mL) was cooled in an ice bath. A base (1.2 equiv) was added to this cooled suspension and stirred continuously. Compound of Formula III was added dropwise to this solution and stirred continuously. The mixture was diluted with the chloroform and the solution was washed with water and brine. The chloroform layer was dried over anhydrous sodium sulfate and concentrated to afford the crude product. The crude product was purified by column chromatography using petroleum ether-ethylacetate mixture to afford the desired product.

The following compounds were prepared by following the above general procedure:

### Compound No. 13: (S)—N-((3-(4-(3-Fluoro-4-(1′-formamido-2′,2′,2′-trichloroethyl)-1-piperazinyl)phenyl)-2-oxo-5-oxazolidinyl)methyl)-acetamide

The following spectral identification confirmed product formation et seq. ¹HNMR (200 MHz, CDCl₃): δ 8.47 (s, 1H), 7.39 (dd, J=14.1 Hz, J=2.6 Hz, 1H), 6.85-7.10 (m, 3H), 5.43 (d, 1H), 4.76 (bm, 1H), 4.01 (t, J=9 Hz, 1H), 3.55-3.80 (m, 4H), 2.80-3.75 (m, 8H), 2.02 (s, 3H)

### Compound No. 14: (S)—N-((3-(4-(3-Fluoro-4-(1′-acetamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 7.36 (dd, J=14.1 Hz, J=2.6 Hz, DH), 7.05 (d, J=8.8 Hz, 1H), 6.75-6.90 (m, 1H), 6.58 (bt, J=9 Hz, 1H), 5.35 (d, 1H), 4.77 (bm, 1H), 4.01 (t, J=9 Hz, 1H), 3.55-3.80 (m, 4H), 2.90-3.15 (m, 8H), 2.03 (s, 3H)

### Compound No. 15: (S)—N-((3-(4-(3-Fluoro-4-(1′-trichloroacetamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 8.10 (s, 1H), 7.47 (dd, J=14.1 Hz, J=2.6 Hz, 1H), 7.05 (d, J=8 Hz, 1H), 6.92 (t, 1H), 6.25 (bt, J=11 Hz, 1H), 4.78 (bt, 1H), 4.03 (t, J=9 Hz, 1H), 3.55-3.80 (m, 4H), 2.90-3.15 (m, 4H), 2.03 (s, 3H)

### Compound No. 16: (S)—N-((3-(4-(3-Fluoro-4-(1′-trifluoroacetamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 8.09 (s, 1H), 7.47 (dd, J=14.1 Hz, J=2.6 Hz, 1H), 7.07 (d, J=8 Hz, 1H), 6.92 (t, 1H), 6.39 (bt, J=11 Hz, 1H), 5.30 (d, J=1H), 4.79 (bm, 1H), 4.02 (t, J=9 Hz, 1H), 3.59-3.80 (m, 8H), 2.95-3.15 (m, 4H), 2.02 (s, 3H)

### Compound No. 17: (S)—N-((3-(4-(3-Fluoro-4-(1′-formamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 8.48 (s, 1H), 7.39 (dd, J=14.1 Hz, J=2.6 Hz, 1H), 7.04 (d, J=8 Hz, 1H), 6.92 (t, 1H), 6.40 (bt, J=11 Hz, 1H), 5.35 (d, 1H), 4.76 (bm, 1H), 4.03 (t, J=9 Hz, 1H), 3.55-3.80 (m, 4H), 2.89-3.30 (m, 8H), 2.02 (s, 3H)

### Compound No. 18: (S)—N-((3-(4-(3-Fluoro-4-(1′-acetamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 7.41 (dd, J=14.1 Hz, J=2.6 Hz, 1H), 7.07 (d, J=8 Hz, 1H), 6.91 (t, 1H), 6.34 (t, 1H), 5.32 (d, 1H), 4.79 (bm, 1H), 4.02 (t, J=9 Hz, 1H), 3.60-3.86 (m, 4H), 2.85-3.30 (m, 8H), 2.19 (s, 3H), 2.03 (s, 3H)

### Compound No. 19: (S)—N-((3-(4-(3-Fluoro-4-(1′-trichloroacetamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 8.09 (s, 1H), 7.47 (dd, J=14.1 Hz, J=2.6 Hz, 1H), 7.07 (d, J=8 Hz, 1H), 6.91 (t, 1H), 6.37 (bt, J=11 Hz, 1H), 4.78 (bm, 1H), 4.02 (t, 1H), 3.50-3.85 (m, 8H), 2.95-3.20 (m, 84), 2.02 (s, 3H)

### Compound No. 20: (S)—N-((3-(4-(3-Fluoro-4-(1′-trifluoroacetamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 8.10 (s, 1H), 7.47 (dd, J=14.1 Hz, J=2.6 Hz, 1H), 6.85-7.15 (m, 2H), 6.31 (t, 1H), 4.78 (bm, 1H), 4.02 (t, J=9 Hz, 1H), 3.45-3.85 (m, 8H), 2.90-3.10 (m, 4H), 2.02 (s, 3H)

### Compound No. 21: (S)—N-((3-(4-(3-Fluoro-4-(1′-formamido-2′,2′,2′-trifluoroethyl)-1-piperazinyl)phenyl)-2-oxo-5-oxazolidinyl)methyl)-acetamide

The following spectral identification confirmed product formation. ¹HNMR (200 MHz, CDCl₃): δ 8.43 (s, 1H0, 7.39 (dd, J=14.1 Hz, J=2.6 Hz, 1H), 7.04 (d, J=8.8 Hz, 1H), 7.06 (d, J=8 Hz, 1H), 6.90 (t, J=9 Hz, 1H), 6.47 (d, J=9 Hz, 1H), 5.46 (bt, 1H), 4.77 (bm, 1H), 4.02 (t, J=9 Hz, 1H), 3.60-3.85 (m, 4H), 2.70-3.15 (m, 8H), 2.02 (s, 3H)

### Compound No. 22: (S)—N-((3-(4-(3-Fluoro-4-(1′-acetamido-2′,2′,2′-trifluoroethyl)-1-piperazinyl)phenyl)-2-oxo-5-oxazolidinyl)methyl)-acetamide

The following spectral identification confirmed product formation. ¹HNMR (200 MHz, CDCl₃): δ 7.42 (dd, J=14.1 Hz, J=2.6 Hz, 1H), 7.05 (d, J=8 Hz, 1H), 6.91 (t, J=9 Hz, 1H), 6.14 (bt, J=11 Hz, 1H), 5.39 (bt, 1H0, 4.74 (bm, 1H), 4.02 (t, J=9 Hz, 1H), 3.55-3.80 (m, 4H), 2.75-3.15 (m, 8H), 2.13 (s, 1H), 2.03 (s, 3H)

### Compound No. 23: (S)—N-((3-(4-(3-Fluoro-4-(1′-formamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 8.08 (bs, IH), 7.45 (dd, J=14.1 Hz, J=2.6 Hz, 1H), 7.08 (d, J=8 Hz, 1H), 6.90 (t, 1H), 4.86 (bm, 1H), 4.11 (t, J=9 Hz, 1H), 3.59-3.80 (m, 8H), 2.90-3.20 (m, 4H)

### Compound No. 24: (S)—N-((3-(4-(3-Fluoro-4-(1′-acetamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 7.41 (dd, J=14.1 Hz, J=2.6 Hz, 1H), 7.05 (d, J=8 Hz, 1H), 6.93 (t, 1H), 6.11 (dd, J=14 Hz, J=2.6 Hz, 1H), 5.35 (d, 1H), 4.85 (bm, 1H), 4.11 (t, 1H), 3.60-3.95 (m, 4H), 2.80-3.20 (m, 8H), 2.20 (s, 3H)

### Compound No. 25: (S)—N-((3-(4-(3-Fluoro-4-(1′-trichloroacetamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 8.10 (s, 1H), 7.30-7.50 (m, 2H), 7.07 (d, J=8 Hz, 1H), 6.92 (t, 1H), 4.83 (bm, 1H), 4.11 (t, J=9 Hz, 1H), 3.50-3.95 (m, 8H), 2.90-3.15 (m, 4H)

### Compound No. 26: (S)—N-((3-(4-(3-Fluoro-4-(1′-trifluoroacetamido-2′,2′,2′-trichloroethyl)-1-piperazinyl)phenyl)-2-oxo-5-oxazolidinyl)methyl)-acetamide

The following spectral identification confirmed product formation. ¹HNMR (200 MHz, CDCl₃): δ 8.09 (s, IH), 7.72 (dd, J=14.1 Hz, J=2.6 Hz, 1H), 7.08 (d, J=8 Hz, 1H), 6.92 (t, 1H), 4.85 (bm, 1H), 4.11 (t, 1H), 3.55-3.95 (m, 8H), 2.90-3.20 (m, 4H)

### Compound No. 27: (S)—N-((3-(4-(3-Fluoro-4-(1′-formamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 8.48 (s, IH), 7.47 (dd, J=14.1 Hz, J=2.6 Hz, 1H), 7.05-7.20 (m, 2H), 6.38 (bm, 1H), 5.41 (d, 1H), 4.85 (bm, 1H), 4.13 (t, J=9 Hz, 1H), 3.65-3.95 (m, 4H), 3.00-3.45 (m, 8H)

### Compound No. 28: (S)—N-((3-(4-(3-Fluoro-4-(1′-acetamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 6.910-7.45 (m, 4H), 6.10 (d, 1H), 5.32 (d, 1H), 4.83 (bm, 1H), 4.11 (t, J=9 Hz, 1H), 3.55-3.80 (m, 4H), 2.75-3.15 (m, 8H), 2.13 (s, 3H), 2.03 (s, 3H)

### Compound No. 29: (S)—N-((3-(4-(3-Fluoro-4-(1′-trichloroacetamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 8.62 (s, 1H), 7.47 (dd, J=14.1 Hz, J=2.6 Hz, 1H), 7.05-7.20 (m, 2H), 6.95 (t, 1H), 4.85 (bm, 1H), 3.60-4.20 (m, 8H), 3.05-3.25 (m, 4H)

### Compound No. 30: (S)—N-((3-(4-(3-Fluoro-4-(1′-trifluoroacetamido-2′,2′,2′-tribromoethyl)-1-piperazinyl)phenyl)-2-oxo-5-oxazolidinyl)methyl)-acetamide

The following spectral identification confirmed product formation. ¹HNMR (200 MHz, CDCl₃): δ 8.10 (s, 1H), 7.35-7.65 (m, 2H), 7.08 (d, J=8 Hz, 1H), 6.92 (t, 1H), 4.85 (bm, 1H), 4.11 (t, J=9 Hz, 1H), 3.50-3.90 (m, 8H), 2.90-3.10 (m, 4H)

### Compound No. 31: (S)—N-((3-(4-(3-Fluoro-4-(1′-formamido-2′,2′,2′-trifluoroethyl)-1-piperazinyl)phenyl)-2-oxo-5-oxazolidinyl)methyl)-acetamide

The following spectral identification confirmed product formation. ¹HNMR (200 MHz, CDCl₃): δ 8.09 (s, 1H), 7.57 (t, 1H), 7.43 (dd, J=14.1 Hz, J=2.6 Hz, 1H), 7.10 (d, J=8 Hz, 1H), 6.92 (t, 1H), 4.87 (bm, 1H), 4.11 (t, J=9 Hz, 1H), 3.55-3.95 (m, 8H), 2.90-3.25 (m, 4H)

### Compound No. 32: (S)—N-((3-(4-(3-Fluoro-4-(1′-acetamido-2′,2′,2′-trifluoroethyl)-1-piperazinyl)phenyl)-2-oxo-5-oxazolidinyl)methyl)-acetamide

The following spectral identification confirmed product formation. ¹HNMR (200 MHz, CDCl₃): δ 7.39 (dd, J=14.1 HZ, J=2.6 Hz, 2H), 7.05 (d, J=8 Hz, 1H), 6.92 (t, 1H), 5.39 (d, 1H), 4.82 (bm, 1H), 4.11 (t, J=9 Hz, 1H), 3.55-3.95 (m, 4H), 2.75-3.20 (m, 8H), 2.13 (s, 3H)

### Compound No. 33: (S)—N-((3-(4-(3-Fluoro-4-(1′-trichloroacetamido-2′,2′,2′-trifluoroethyl)-1-piperazinyl)phenyl)-2-oxo-5-oxazolidinyl)methyl)-acetamide

The following spectral identification confirmed product formation. ¹HNMR (200 MHz, CDCl₃): δ 6.85-7.60 (m, 4H), 5.53 (m, 1H), 4.85 (bm, 1H), 4.11 (t, J=9 Hz, 1H), 3.60-3.95 (m, 4H), 2.65-3.20 (m, 6H)

### Compound 34: (S)—N-((3-(4-(3-Fluoro-4-(1′-formamido-2′,2′,2′-trichloroethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 8.46 (s, 1H), 7.43 (dd, J=14.1 Hz, J=2.6 Hz, 1H), 7.35 (dd, J=14.1 Hz, J=2.6 Hz, 1H), 7.0-7.15 (m, 1H), 6.88 (t, J=9.1 Hz, 1H), 6.68 (d, J=4 Hz, 1H), 6.45 (t, 1H), 5.83 (d, 1H), 4.77 (bm, 1H), 4.01 (t, J=9 Hz, 1H), 3.60-3.85 (m, 4H), 3.15-3.55 (m, 4H), 2.60-3.10 (m, 4H), 2.02 (s, 3H), 1.34 (d, 3H)

### Compound 35: (S)—N-((3-(4-(3-Fluoro-4-(1′-acetamido-2′,2′,2′-trichloroethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 7.30-7.50 (m, 1H), 7.0-7.10 (m, 1H), 6.87 (t, J=9.1 Hz, 1H), 6.45 (t, 1H), 6.20-6.35 (m, 1H), 5.43 (d, 1H), 4.76 (m, 1H), 4.01 (t, J=9 Hz, 1H), 3.60-3.85 (m, 4H), 2.85-3.50 (m, 8H), 2.14 (s, 3H), 2.02 (s, 3H), 1.31 (d,), 1.25 (d)

### Compound 36: (S)—N-((3-(4-(3-Fluoro-4-(1′-trichloroacetamido-2′,2′,2′-trichloroethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 8.17 (s), 8.05 (s), 7.46 (dd, J=14 Hz, J=2.6 Hz, 1H), 7.05 (d, J=8 Hz, 1H), 6.90 (t, J=9.1 Hz, 1H), 6.49 (t, 1H), 4.78 (m, 1H), 4.02 (t, J=9 Hz, 1H), 3.15-3.85 (m, 7H), 2.75-3.0 (m, 2H), 2.03 (s, 3H), 1.40 (d)

### Compound 37: (S)—N-((3-(4-(3-Fluoro-4-(1′-trifluoroacetamido-2′,2′,2′-trichloroethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 8.17 (s), 8.05 (s), 7.46 (dd, J=14 Hz, J=2.6 Hz, 1H), 7.05 (d, J=8 Hz, 1H), 6.90 (t, J=9.1 Hz, 1H), 6.49 (t, 1H), 4.78 (m, 1H), 4.02 (t, J=9 Hz, 1H), 3.15-3.85 (m, 7H), 2.75-3.0 (m, 2H), 2.03 (s, 3H), 1.50 (d), 1.40 (d)

### Compound 38: (S)—N-((3-(4-(3-Fluoro-4-(1′-formamido-2′,2′,2′-tribromoethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 8.17 (s), 8.05 (s), 7.46 (dd, J=14 Hz, J=2.6 Hz, 1H), 7.07 (d, J=8 Hz, 1H), 6.89 (t, J=9.1 Hz, 1H), 6.19 (t, 1H), 4.75 (m, 1H), 4.03 (t, J=9 Hz, 1H), 3.60-3.80 (m, 6H), 3.20-3.50 (m, 5H), 2.70-3.0 (m, 3H), 2.03 (s, 3H), 1.50 (d), 1.42 (d)

### Compound 39: (S)—N-((3-(4-(3-Fluoro-4-(1′-acetamido-2′,2′,2′-tribromoethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 8.39 (s), 7.41 (dd, J=14 Hz, J=2.6 Hz, 1H), 6.85-7.10 (m, 2H), 6.15-6.45 (m, 2H), 5.46 (d, 1H), 4.77 (m, 1H), 3.95-4.20 (m, 2H), 3.45-3.85 (m, 4H), 2.90-3.35 (m, 6H), 2.03 (s, 3H), 1.36 (d), 1.25 (d)

### Compound 40: (S)—N-((3-(4-(3-Fluoro-4-(1′-trichloroacetamido-2′,2′,2′-tribromoethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 7.39 (dd, J=14 Hz, J=2.6 Hz, 1H), 7.05 (d, J=8 Hz, 1H), 6.87 (t, J=9.1 Hz, 1H), 6.35 (m, 1H), 6.11 (m, 1H), 5.37 (d, 1H), 4.77 (m, 1H), 4.0 (t, J=9 Hz, 1H), 3.45-3.85 (m, 4H), 3.05-3.30 (m, 6H), 2.17 (s, 3H), 2.03 (s, 3H), 1.45 (d), 1.30 (d)

### Compound 41: (S)—N-((3-(4-(3-Fluoro-4-(1′-trifluoroacetamido-2′,2′,2′-tribromoethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 8.17 (s), 8.05 (s), 7.44 (dd, J=14 Hz, J=2.6 Hz, 1H), 7.07 (d, J=8 Hz, 1H), 6.89 (t, J=9.1 Hz, 1H), 6.31 (t, 1H), 4.76 (m, 1H), 4.02 (t, J=9 Hz, 1H), 3.55-3.80 (m, 3H), 3.15-3.50 (m, 3H), 2.20-2.80 (m, 2H), 2.02 (s, 3H), 1.50 (d), 1.41 (d)

### Compound 42: (S)—N-((3-(4-(3-Fluoro-4-(1′-formamido-2′,2′,2′-trifluoroethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 7.42 (dd, J=14 Hz, J=2.6 Hz, 1H), 7.05 (d, J=8 Hz, 1H), 6.93 (t, J=9.1 Hz, 1H), 6.24 (t, 1H), 4.76 (m, 1H), 4.02 (t, J=9 Hz, 1H), 3.60-3.80 (m, 4H), 3.05-3.35 (m, 6H), 2.70-2.85 (m, 1H), 2.03 (s, 3H), 1.16 (d, 3H)

### Compound 43: (S)—N-((3-(4-(3-Fluoro-4-(1′-acetamido-2′,2′,2′-trifluorethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 8.40 (s), 8.31 (s), 7.40 (dd, J=14 Hz, J=2.6 Hz, 1H), 7.05 (d, J=8 Hz, 1H), 6.80-6.95 (m, 1H), 6.60 (d, J=8 Hz, 1H), 6.30 (m, 1H), 4.77 (m, 1H), 4.02 (t, J=9 Hz, 1H), 3.65-3.85 (m, 4H), 2.75-3.35 (m, 7H), 2.02 (s, 3H), 1.29 (d), 1.26 (d)

### Compound 44: (S)—N-((3-(4-(3-Fluoro-4-(1′-trichloroacetamido-2′,2′,2′-trifluoroethyl)-3-methyl-1-piperazinyl)phenyl)-2-oxo-5-oxazolidinyl)methyl)-acetamide

The following spectral identification confirmed product formation. ¹HNMR (200 MHz, CDCl₃): δ 7.42 (dd, J=14 Hz, J=2.6 Hz, 1H), 7.04 (d, J=8 Hz, 1H), 6.91 (t, J=9.1 Hz, 1H), 6.15-6.35 (m, 2H), 4.77 (m, 1H), 4.02 (t, J=9 Hz, 1H), 3.55-3.80 (m, 3H), 2.55-3.35 (m, 7H), 2.11 (s), 2.02 (s), 1.27 (d)

### Compound 45: (S)—N-((3-(4-(3-Fluoro-4-(1′-formamido-2′,2′,2′-trichloroethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃): δ 7.43 (dd, J=14 Hz, J=2.6 Hz, 1H), 7.02-7.23 (m, 2H), 6.91 (t, J=9.1 Hz, 1H), 6.34 (t, 1H), 4.76 (m, 1H), 4.02 (t, J=9 Hz, 1H), 3.55-3.80 (m, 4H), 2.80-3.40 (m, 7H), 2.02 (s, 3H), 1.30 (d), 1.26 (d)

### Compound 46: (S)—N-((3-(4-(3-Fluoro-4-(1′-acetamido-2′,2′,2′-trichloroethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃): δ 8.47 (s), 8.37 (s), 7.30-7.45 (m, 2H), 7.04 (d, J=8 Hz, 1H), 6.90 (t, J=9.1 Hz, 1H), 6.44 (t, 1H), 5.84 (d), 4.84 (m, 1H), 4.11 (t, J=9 Hz, 1H), 2.60-3.90 (m, 11H), 1.35 (d, 3H)

### Compound 47: (S)—N-((3-(4-(3-Fluoro-4-(1′-trichloroacetamido-2′,2′,2′-trichloroethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃): δ 7.62 (bm, 1H), 7.30-7.40 (m, 1H), 7.05 (d, J=8 Hz, 1H), 6.92 (t, J=9.1 Hz, 1H), 6.19 (d, 1H), 5.44 (d, 1H), 4.85 (bm, 1H), 4.10 (t, J=9 Hz, 1H), 2.90-3.90 (m, 10H), 2.15 (s, 3H), 1.3 (d), 1.27 (d)

### Compound 48: (S)—N-((3-(4-(3-Fluoro-4-(1′-trifluoroacetamido-2′,2′,2′-trichloroethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃): δ 8.17 (s), 8.05 (s), 7.42 (dd, J=14 Hz, J=2.6 Hz, 1H), 7.07 (d, J=8 Hz, 1H), 6.90 (t, J=9.1 Hz, 1H), 4.83 (m), 4.68 (bm), 4.11 (t, J=9 Hz, 1H), 2.70-3.95 (m, 10H), 1.51 (d), 1.41 (d)

### Compound 49: (S)—N-((3-(4-(3-Fluoro-4-(1′-formamido-2′,2′,2′-tribromoethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃): δ 8.16 (s), 8.05 (s), 7.35-7.60 (m), 7.06 (d, J=8 Hz, 1H), 6.90 (t, J=9.1 Hz, 1H), 4.85 (bm), 4.67 (bm), 4.11 (t, J=9 Hz, 1H), 2.70-3.90 (bm, 10H), 1.51 (d), 1.41 (d)

### Compound 50: (S)—N-((3-(4-(3-Fluoro-4-(1′-acetamido-2′,2′,2′-tribromoethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃): δ 7.70-7.85 (m, 1H), 7.43 (dd, J=14 Hz, J=2.6 Hz, 1H), 7.25 (m, 1H), 7.08 (m, 1H), 6.96 (t, J=9.1 Hz, 1H), 5.53 (s), 4.85 (bm, 1H), 3.18-3.95 (bm, 11H), 2.19 (s, 3H), 1.51 (d), 1.41 (d)

### Compound 51: (S)—N-((3-(4-(3-Fluoro-4-(1′-trichloroacetamido-2′,2′,2′-tribromoethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃): δ 8.16 (s), 8.04 (s), 7.70 (bm, 1H), 7.41 (dd, J=14 Hz, J=2.6 Hz, 1H), 7.05 (d, J=8 Hz, 1H), 6.90 (t, J=9.1 Hz, 1H), 4.85 (bm), 4.66 (bm), 4.11 (t, J=9 Hz, 1H), 3.15-3.90 (bm, 9H), 2.65-3.0 (bm, 3H), 1.51 (d), 1.41 (d)

### Compound 52: (S)—N-((3-(4-(3-Fluoro-4-(1′-trifluoroacetamido-2′,2′,2′-tribromoethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃-CD₃OD): δ 7.30-7.50 (m, 2H), 6.90-7.15 (m, 2H), 4.84 (bm, 1H), 4.11 (t, J=9 Hz, 1H), 2.90-3.85 (m), 1.45 (d)

### Compound 53: (S)—N-((3-(4-(3-Fluoro-4-(1′-formamido-2′,2′,2′-trifluoroethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃): δ 8.33 (s), 8.23 (s), 8.17 (s), 7.35-7.45 (m, 1H), 6.85-7.05 (m, 2H), 5.74 (d), 5.53 (d), 4.81 (bm, 1H), 4.09 (t, J=9 Hz, 1H), 3.55-3.85 (m, 3H), 2.70-3.35 (m, 8H), 1.27 (d), 1.25 (d)

### Compound 54: (S)—N-((3-(4-(3-Fluoro-4-(1′-acetamido-2′,2′,2′-trifluoroethyl)-3-methyl-1-piperazinyl)phenyl)-2-oxo-5-oxazolidinyl)methyl)-trifluoroacetamide

The following spectral identification confirmed product formation. ¹HNMR (200 MHz, CDCl₃) δ 8.81 (bs), 7.35 (dd, J=14 Hz, J=2.6 Hz, 1H), 7.04 (d, J=8 Hz, 1H), 6.89 (t, J=9.1 Hz, 1H), 5.10 (d), 4.83 (bm, 1H), 4.08 (t, J=9 Hz, 1H), 3.55-3.85 (m, 3H), 2.70-3.30 (m, 8H), 2.07 (s, 3H), 1.26 (d, 3H)

### Pharmacological Testing

The compounds of the invention display antibacterial activity when tested under the methods described below. Table II discloses minimum inhibitory concentrations (μg/mL) obtained for the representative compounds of the invention.

### Medium

a) Cation adjusted Mueller Hinton Agar (MHA-Difco)

### b) Trypticase Soya Agar (TSA)

### Inoculum Preparation

Inoculum was prepared by streaking cultures on TSA for aerobic cultures and MHA with 5% sheep blood for fastidious cultures. Aerobic cultures and fastidious cultures were incubated in a CO₂ incubator (5% CO₂) for 18-24 hours at 37° C. Three to four well-isolated colonies were taken and saline suspensions were prepared in sterile densimat tubes. The turbidity of the culture was adjusted to 0.5-0.7 McFarland standard (1.5×10⁸ CFU/mL). The cultures were diluted 10-fold in saline to provide an inoculum size of approximately 1-2×10⁷ organisms/mL.

### Preparation of Active Compound at Desired Concentration

A 1 mg/mL stock solution of active compound in DMSO/distilled water/solvent was prepared and a series of active compound solutions having various concentrations was prepared by two-fold serial dilutions according to procedures disclosed in the NCCLS manual (National Committee for Clinical Laboratory Standards (NCCLS), Methods for Dilution Antimicrobial Susceptibility Tests for Bacteria That Grow Aerobically—Fifth Edition; Approved Standard. M7-A5, Vol. 20. No. 2 (January 2000)).

The stock solution was changed according to the need of the experiment.

### Preparation of Agar Plates

2 mL of each active compound solution in the series was added to 18 mL of Molten Mueller Hinton agar before solidification. Each mixture in the series contained the active compound solution within a required concentration range, for example, 0.015 μg/mL-16 μg/mL. 1 mL of sheep blood was added to the Molten Mueller Hinton agar for fastidious cultures.

MHA and MHA with 5% sheep blood plates without antibiotic were prepared as controls for each set. Additional MHA and MHA with 5% sheep blood plates without antibiotic were prepared in determining quality check for media.

### Preparation of Teflon Template

1 μL of each culture was replicated with the help of a replicator (Denley's multipoint replicator). Culture spots were dried and the plates were incubated for 18-24 hours at 37° C. Fastidious culture spots were incubated at 37° C. in a CO₂ incubator. The results were then compared with the control plates.

### Endpoint Definition

The concentration of drug at which there is complete disappearance of growth spot or formation of less than 10 colonies per spot was considered the minimum inhibitory concentration (MIC). The quality control strains were read and plotted on a Quality Control (QC) chart for agar dilution method. The concentration showing no growth of the inoculated culture was recorded as the MIC. Appropriate ATCC standard (quality control) strains (e.g., S. aureus ATCC 29213, E. faecalis ATCC 29212, E. coli ATCC 25922, and P. aeruginosa ATCC 27853) were simultaneously tested and the results were recorded only when the MIC's against standard antibiotics are within the acceptable range.

Media Control: NCCLS disc diffusion assay using 10 μg discs of Gentamicin (Difco) against Pseudomonas aeruginosa ATCC 27853. A zone diameter of 16-21 mm was considered for optimum cation (Mg and Ca) content of the media. The diameter was plotted in a media QC chart.

### Results

The compounds disclosed herein were found to be highly active against staphylococci, enterococci and S. pneumoniae strains. MIC of the disclosed compounds were 0.5-2 μg/mL for staphylococci, including methecin resistant Staphylococcus aureus (MRSAs); 1-2 μg/mL for enterococci including vancomycin resistant enterococci (VREs); 0.25-2 μg/mL for S. pneumoniae strains including DRSP (Drug resistant streptococcus pneumoniae) and 0.5-2 μg/mL for S. pyogenes

### REFERENCES

a. National Committee for Clinical Laboratory Standards (NCCLS), Methods for Dilution Antimicrobial Susceptibility Tests for Bacteria That Grow Aerobically—Fifth Edition; Approved Standard. M7-A5, Vol. 20. No. 2 (January 2000).
b. National Committee for Clinical Laboratory Standards, Performance Standards for Antimicrobial Susceptibility Testing—Twelfth informational supplement, M 100-S12, Vol. 22 No. 1 (January 2002).

We Claim:

1. A compound having the structure of Formula I: 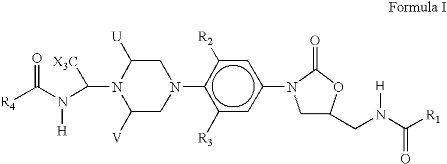
and its pharmaceutically acceptable salts, pharmaceutically acceptable solvates, enantiomers, diasteromers, N-oxides or polymorphs wherein:
R₁ and R₄ are independently selected from the group consisting of hydrogen; optionally substituted alkyl, alkenyl, alkynyl, or cycloalkyl (wherein the optional substituent is selected from one or more of halogen, hydroxyl or alkoxy); and optionally substituted aryl or heterocycle (wherein the optional substitution is selected from one or more of halogen, hydroxy, mercapto, alkoxy, alkyl, acyl, acyloxy, haloalkyl, amino, cyano, nitro, thio, or thioalkyl);
R₂ and R₃ are independently selected from the group consisting of hydrogen, halogen, alkyl, and haloalkyl;
U and V are independently selected from the group consisting of hydrogen, alkyl, and haloalkyl; and
X is halogen.

2. (canceled)

3. The compound of claim 1, wherein R₁ is methyl or trifluoromethyl.

4. The compound of claim 1, wherein R₃ is halogen.

5. (canceled)

6. The compound of claim 1, wherein U is alkyl.

7. (canceled)

8. The compound of claim 1, wherein X is halogen.

9. (canceled)

10. The compound of claim 1, wherein R₄ is alkyl or aryl optionally substituted with halogen.

11. (canceled)

12. A compound selected from the group consisting of
N-((3-(4-(3-Fluoro-4(1′-formamido-2′,2′,2′-trichloroethyl)-1-piperazinyl)phenyl)-2-oxo-5-oxazolidinyl)methyl)-acetamide,
N-((3-(4-(3-Fluoro-4(1′-acetamido-2′,2′,2′-trichloroethyl)-1-piperazinyl)phenyl)-2-oxo-5-oxazolidinyl)methyl)-acetamide,
N-((3-(4-(3-Fluoro-4(1′-trichloroacetamido-2′,2′,2′-trichloroethyl)-1-piperazinyl)phenyl)-2-oxo-5-oxazolidinyl)methyl)-acetamide,
N-((3-(4-(3-Fluoro-4(1′-trifluoroacetamido-2′,2′,2-trichloroethyl)-1-piperazinyl)phenyl)-2-oxo-5-oxazolidinyl)methyl)-acetamide,
N-((3-(4-(3-Fluoro-4(1′-formamido-2′,2′,2′-tribromoethyl)-1-piperazinyl)phenyl)-2-oxo-5-oxazolidinyl)methyl)-acetamide,
N-((3-(4-(3-Fluoro-4(1′-acetamido-2′,2′,2′-tribromoethyl)-1-piperazinyl)phenyl)-2-oxo-5-oxazolidinyl)methyl)-acetamide,
N-((3-(4-(3-Fluoro-4(1′-trifluoroacetamido-2′,2′,2′-tribromoethyl)-1-piperazinyl)phenyl)-2-oxo-5-oxazolidinyl)methyl)-acetamide,
N-((3-(4-(3-Fluoro-4(1′-benzamido-2′,2′,2′-trichloroethyl)-1-piperazinyl)phenyl)-2-oxo-5-oxazolidinyl)methyl)-acetamide,
N-((3-(4-(3-Fluoro-4(1′-2,4-dichloro-5-fluorobenzamido-2′,2′,2′-trichloroethyl)-1-piperazinyl)phenyl)-2-oxo-5-oxazolidinyl)methyl)-acetamide,
N-((3-(4-(3-Fluoro-4(1′-benzamido-2′,2′,2′-tribromoethyl)-1-piperazinyl)phenyl)-2-oxo-5-oxazolidinyl)methyl)-acetamide,
N-((3-(4-(3-Fluoro-4(1′-2,4-dichloro-5-fluorobenzamido-2′,2′,2′-tribromoethyl)-1-piperazinyl)phenyl)-2-oxo-5-oxazolidinyl)methyl)-acetamide.
N-((3-(4-(3-Fluoro-4(1′-formamido-2′,2′,2′-trifluoroethyl)-1-piperazinyl)phenyl)-2-oxo-5-oxazolidinyl)methyl)acetamide,
(S)—N-((3-(4-(3-Fluoro-4(1′-formamido-2′,2′,2′-trichloroethyl)-1-piperazinyl)phenyl)-2-oxo-5-oxazolidinyl)methyl)-acetamide,
(S)—N-((3-(4-(3-Fluoro-4(1′-acetamido-2′,2′,2′-trichloroethyl)-1-piperazinyl)phenyl)-2-oxo-5-oxazolidinyl)methyl)-acetamide,
(S)—N-((3-(4-(3-Fluoro-4(1′-trichloroethyl)-1-piperazinyl)phenyl)-2-oxo-5-oxazolidinyl)methyl)-acetamide,
(S)—N-((3-(4-(3-Fluoro-4(1′-trifluoroacetamido-2′,2′,2′-trichloroethyl)-1-piperazinyl)phenyl)-2-oxo-5-oxazolidinyl)methyl)-acetamide,
(S)—N-((3-(4-(3-Fluoro-4(1′-formamido-2′,2′,2′-tribromoethyl)-1-piperazinyl)phenyl)-2-oxo-5-oxazolidinyl)methyl)-acetamide,
(S)—N-((3-(4-(3-Fluoro-4(1′-acetamido-2′,2′,2′-tribromoethyl)-1-piperazinyl)phenyl)-2-oxo-5-oxazolidinyl)methyl)-acetamide,
(S)—N-((3-(4-(3-Fluoro-4(1′-trichloroacetamido-2′,2′,2′-tribromoethyl)-1-piperazinyl)phenyl)-2-oxo-5-oxazolidinyl)methyl)-acetamide,
(S)—N-((3-(4-(3-Fluoro-4(1′-trifluoroacetamido-2′,2′,2′-tribromoethyl)-1-piperazinyl)phenyl)-2-oxo-5-oxazolidinyl)methyl)-acetamide,
(S)—N-((3-(4-(3-Fluoro-4(1′-formamido-2′,2′,2′-trifluoroethyl)-1-piperazinyl)phenyl)-2-oxo-5-oxazolidinyl)methyl)-acetamide,
(S)—N-((3-(4-(3-Fluoro-4(1′-acetamido-2′,2′,2′-trifluoroethyl)-1-piperazinyl)phenyl)-2-oxo-5-oxazolidinyl)methyl)-acetamide,
(S)—N-((3-(4-(3-Fluoro-4(1′-formamido-2′,2′,2′-trichloroethyl)-1-piperazinyl)phenyl)-2-oxo-5-oxazolidinyl)methyl)-trifluoroacetamide,
(S)—N-((3-(4-(3-Fluoro-4(1′-acetamido-2′,2′,2′-trichloroethyl)-1-piperazinyl)phenyl)-2-oxo-5-oxazolidinyl)methyl)-trifluoroacetamide,
(S)—N-((3-(4-(3-Fluoro-4(1′-trichloroacetamido-1′,2′,2′-trichloroethyl)-1-piperazinyl)phenyl)-2-oxo-5-oxazolidinyl)methyl)-trifluoroacetamide,
(S)—N-((3-(4-(3-Fluoro-4(1′-trifluoroacetamido-2′,2′,2′-trichloroethyl)-1-piperazinyl)phenyl)-2-oxo-5-oxazolidinyl)methyl)-trifluoroacetamide,
(S)—N-((3-(4-(3-Fluoro-4(1′-formamido-2′,2′,2′-trichloroethyl)-1-piperazinyl)phenyl)-2-oxo-5-oxazolidinyl)methyl)-trifluoroacetamide,
(S)—N-((3-(4-(3-Fluoro-4(1′-acetamido-2′,2′,2′-tribromoethyl)-1-piperazinyl)phenyl)-2-oxo-5-oxazolidinyl)methyl)-trifluoroacetamide,
(S)—N-((3-(4-(3-Fluoro-4(1′-trichloroacetamido-2′,2′,2′-tribromoethyl)-1-piperazinyl)phenyl)-2-oxo-5-oxazolidinyl)methyl)-trifluoroacetamide,
(S)—N-((3-(4-(3-Fluoro-4(1′-trifluoroacetamido-2′,2′,2′-tribromoethyl)-1-piperazinyl)phenyl)-2-oxo-5-oxazolidinyl)methyl)-trifluoroacetamide,
(S)—N-((3-(4-(3-Fluoro-4(1′-formamido-2′,2′,2′-trifluoroethyl)-1-piperazinyl)phenyl)-2-oxo-5-oxazolidinyl)methyl)-trifluoroacetamide,
(S)—N-((3-(4-(3-Fluoro-4(1′-acetamido-2′,2′,2′-trifluoroethyl)-1-piperazinyl)phenyl)-2-oxo-5-oxazolidinyl)methyl)-trifluoroacetamide,
(S)—N-((3-(4-(3-Fluoro-4(1′-trichloroacetamido-2′,2′,2′-trifluoroethyl)-1-piperazinyl)phenyl)-2-oxo-5-oxazolidinyl)methyl)-trifluoroacetamide,
(S)—N-((3-(4-(3-Fluoro-4-(1′-formamido-2′,2′,2′-trifluoroethyl)-3-methyl-1-piperazinyl)phenyl)-2-oxo-5-oxazolidinyl)methyl)-acetamide,
(S)—N-((3-(4-(3-Fluoro-4-(1′-acetamido-2′,2′,2′-trichloroethyl)-3-methyl-1-piperazinyl)phenyl)-2-oxo-5-oxazolidinyl)methyl)-acetamide,
(S)—N-((3-(4-(3-Fluoro-4-(1′-trichloroacetamido-2′,2′,2′-trichloroethyl)-3-methyl-1-piperazinyl)phenyl)-2-oxo-5-oxazolidinyl)methyl-acetamide,
(S)—N-((3-(4-(3-Fluoro-4-(1′-trifluoroacetamido-2′,2′,2′-trichloroethyl)-3-methyl-1-piperazinyl)phenyl)-2-oxo-5-oxazolidinyl)methyl)-acetamide,
(S)—N-((3-(4-(3-Fluoro-4-(1′-formamido-2′,2′,2′-tribromoethyl)-3-methyl-1-piperazinyl)phenyl)-2-oxo-5-oxazolidinyl)methyl)-acetamide,
(S)—N-((3-(4-(3-Fluoro-4-(1′-acetamido-2′,2′,2′-tribromoethyl)-3-methyl-1-piperazinyl)phenyl)-2-oxo-5-oxazolidinyl)methyl)-acetamide,
(S)—N-((3-(4-(3-Fluoro-4-(1′-trichloroacetamido-2′,2′,2′-tribromoethyl)-3-methyl-1-piperazinyl)phenyl)-2-oxo-5-oxazolidinyl)methyl)-acetamide,
(S)—N-((3-(4-(3-Fluoro-4-(1′-trifluoroacetamido-2′,2′,2′-tribromoethyl)-3-methyl-1-piperazinyl)phenyl)-2-oxo-5-oxazolidinyl)methyl)-acetamide,
(S)—N-((3-(4-(3-Fluoro-4-(1′-formamaido-2′,2′,2′-trifluoroethyl)-3-methyl-1-piperazinyl)phenyl)-2-oxo-5-oxazolidinyl)methyl)-acetamide,
(S)—N-((3-(4-(3-Fluoro-4-(1′-acetamido-2′,2′,2′-trifluorethyl)-3-methyl-1-piperazinyl)phenyl)-2-oxo-5-oxazolidinyl)methyl)-acetamide,
(S)—N-((3-(4-(3-Fluoro-4-(1′-trichloroacetamido-2′,2′,2′-trifluoroethyl)-3-methyl-1-piperazinyl)phenyl)-2-oxo-5-oxazolidinyl)methyl)-acetamide,
(S)—N-((3-(4-(3-Fluoro-4-(1′-formamaido-2′,2′,2′-tricholroethyl)-3-methyl-1-piperazinyl)phenyl)-2-oxo-5-oxazolidinyl)methyl)-trifluoroacetamide,
(S)—N-((3-(4-(3-Fluoro-4-(1′-acetamido-2′,2′,2′-trichloroethyl)-3-methyl-1-piperazinyl)phenyl)-2-oxo-5-oxazolidinyl)methyl)-trifluoroacetamide,
(S)—N-((3-(4-(3-Fluoro-4-(1′-trichloroacetamido-2′,2′,2′-trichloroethyl)-3-methyl-1-piperazinyl)phenyl)-2-oxo-5-oxazolidinyl)methyl)-trifluoroacetamide,
(S)—N-((3-(4-(3-Fluoro-4-(1′-trifluoroacetamido-2′,2′,2′-trichloroethyl)-3-methyl-1-piperazinyl)phenyl)-2-oxo-5-oxazolidinyl)methyl)-trifluoroacetamide,
(S)—N-((3-(4-(3-Fluoro-4-(1′-formamido-2′,2′,2′-tribromoethyl)-3-methyl-1-piperazinyl)phenyl)-2-oxo-5-oxazolidinyl)methyl)-trifluoroacetamide,
(S)—N-((3-(4-(3-Fluoro-4-(1′-acetamido-2′,2′,2′-tribromoethyl)-3-methyl-1-piperazinyl)phenyl)-2-oxo-5-oxazolidinyl)methyl)-trifluoroacetamide,
(S)—N-((3-(4-(3-Fluoro-4-(1′-trichloroacetamido-2′,2′,2′-tribromoethyl)-3-methyl-1-piperazinyl)phenyl)-2-oxo-5-oxazolidinyl)methyl)-trifluoroacetamide,
(S)—N-((3-(4-(3-Fluoro-4-(1′-trifluoroacetamido-2′,2′,2′-tribromoethyl)-3-methyl-1-piperazinyl)phenyl)-2-oxo-5-oxazolidinyl)methyl)-trifluoroacetamide,
(S)—N-((3-(4-(3-Fluoro-4-(1′-formamido-2′,2′,2′-trifluoroethyl)-3-methyl-1-piperazinyl)phenyl)-2-oxo-5-oxazolidinyl)methyl)-trifluoroacetamide or
(S)—N-((3-(4-(3-Fluoro-4-(1′-acetamido-2′,2′,2′-trifluoroethyl)-3-methyl-1-piperazinyl)phenyl)-2-oxo-5-oxazolidinyl)methyl-trifluoroacetamide,
their pharmaceutically acceptable salts, pharmaceutically acceptable solvates, enantiomers, diasteromers, polymorphs or N-oxides.

13. A pharmaceutical composition comprising a therapeutically effective amount of a compound of claim 1 together with a pharmaceutically acceptable carrier, excipient or diluent.

14. A method of treating or preventing a condition caused by or contributed to by bacterial infection in a mammal comprising administering to the mammal a therapeutically effective amount of a compound of claim 1.

15. A method of treating or preventing a condition caused by or contributed to by bacterial infection in a mammal comprising administering to the mammal a therapeutically effective amount of a pharmaceutical composition of claim 13.

16. The method of claim 14, wherein the condition is selected from the group consisting of community-acquired pneumonia, upper and lower respiratory tract infections, skin and soft tissue infections, bone and joint infections, hospital-acquired lung infections, mastitis, catheter infection and foreign body or prosthesis infections.

17.-18. (canceled)

19. The method of claim 18, wherein the gram-positive or gram-negative bacterium is selected from the group consisting of Staphylococci, Streptococci, Enterococci, Bactericides spp., Clostridium spp., Mycobacterium spp., Bacillus spp., Corynebacterium spp., Peptostrepiococcus spp., Listeria spp., Legionella spp., Haemophilus influenza, Moraxella, Eschericia faecalis and Eschericia coli.

20. The method according to claim 19, wherein said bacterium is a gram-positive coccus.

21. The method according to claim 20, wherein the gram-positive coccus is drag-resistant.

22. A process for preparing a compound of Formula IV, 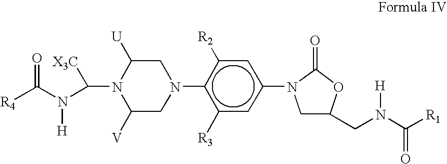
and its pharmaceutically acceptable salts, pharmaceutically acceptable solvates, enantiomers, diasteromers, N-oxides or polymorphs, wherein
R₁ and R₄ are independently selected from the group consisting of hydrogen; optionally substituted alkyl, alkenyl, alkynyl, or cycloalkyl (wherein optional substituent is selected from one or more of halogen, hydroxy or alkoxy); and optionally substituted aryl or heterocycle (wherein the optional substituent is selected from one or more of halogen, hydroxy, mercapto, alkoxy, alkyl, acyl, acyloxy, haloalkyl, amino, cyano, nitro, thio or thioalkyl):
R₂ and R₃ are independently selected from the group consisting of hydrogen, halogen, alkyl, and haloalkyl;
U and V are independently selected from the group consisting of hydrogen, alkyl, and haloalkyl; and
X is halogen,
which method comprises reacting a compound of Formula II with a compound of Formula III

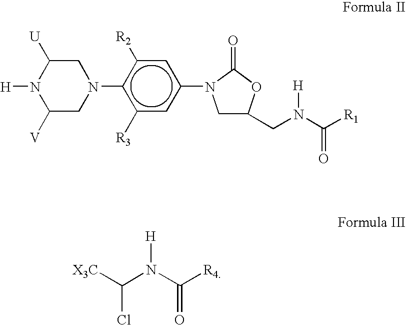

23.-26. (canceled)

27. A process for the preparation of compound of Formula VI, 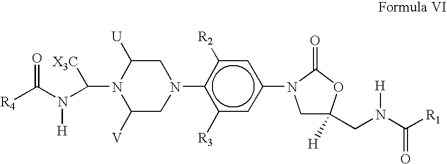
its pharmaceutically acceptable salts, pharmaceutically acceptable solvates, enantiomers, diasteromers, N-oxide or polymorphs, wherein
R₁ and R₄ are independently selected from the group consisting of hydrogen; optionally substituted alkyl, alkenyl, alkynyl, cycloalkyl (wherein the optional substituent is one or more of halogen, hydroxy or alkoxy); optionally substituted aryl or heterocycle (wherein the optional substituent is one or more of halogen, hydroxy, mercapto, alkoxy, alkyl, acyl, acyloxy, haloalkyl, amino, cyano, nitro, thio or thioalkyl);
R₂ and R₃ are independently selected from the group consisting of hydrogen, halogen, alkyl and haloalkyl;
U and V are independently selected from the group consisting of hydrogen, alkyl, haloalkyl; and
X is halogen,
which method comprises reacting a compound of Formula V with a compound of Formula III to give a compound of Formula VI.

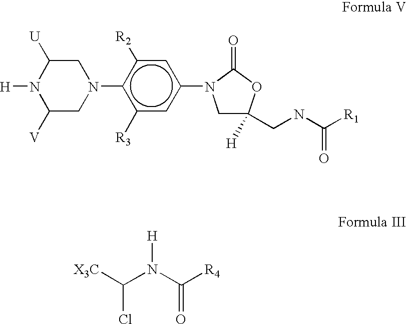

28.-30. (canceled)
